(19) United States
(12) Patent Application Publication
(10) Pub. No.: US 2023/0031549 A1
(43) Pub. Date: Feb. 2, 2023

(54) SUBSTANTIALLY SOLID SOLUTION, SYSTEMS AND METHODS FOR GENERATING A SUBSTANTIALLY SOLID SOLUTION, AND METHODS OF ADMINISTRATION THEREOF

(71) Applicant: MIRAKI INNOVATION THINK TANK LLC, Cambridge, MA (US)

(72) Inventors: Christopher VELIS, Lexington, MA (US); Karen E. MILLER, South Dartmouth, MA (US); Emilia JAVORSKY, Watertown, MA (US); Rainuka GUPTA, Newton, MA (US); Tarik CHAUDHRY, Boston, MA (US)

(73) Assignee: MIRAKI INNOVATION THINK TANK LLC, Cambridge, MA (US)

(21) Appl. No.: 17/788,048

(22) PCT Filed: Dec. 18, 2020
(86) PCT No.: PCT/US20/66093

§ 371 Date: Jun. 22, 2022

Related U.S. Application Data
(60) Provisional application No. 62/953,272, filed on Dec. 24, 2019.

(51) Int. Cl. A61K 9/00; A61K 47/02; A61B 34/10

(52) U.S. Cl. CPC A61K 9/0024; A61K 47/02; A61B 34/10; A61B 2034/105

(57) ABSTRACT

A substantially solid solution is provided, which includes water and optionally one or more additives, wherein about 71% to about 100% of the water by volume is in a solid state. The substantially solid solution may be administered to a subcutaneous fat layer of a subject, in order to reduce or remove adipocytes through freezing. The substantially solid solution may be generated in a mold, or at a point of delivery, and may be administered by any suitable device or administered directly to a treatment site via an incision.

### TECHNICAL FIELD

This disclosure is directed to a substantially solid solution, systems and methods for generating a substantially solid solution, and methods of treatment and removal of subcutaneous fat by administering a substantially solid solution to a subject for fat reduction.

### BACKGROUND

Subcutaneous adipose tissue (SAT, subcutaneous fat) is fat stored just beneath the skin, and is present in varying amounts that generally correlate with genetic and lifestyle factors. Subcutaneous fat helps store energy for the body, provides minor thermoregulation through insulation, and provides a layer of protection for muscles and bones against potential injury through impact. However, subcutaneous fat may impact health, fitness, and appearance, and has been shown to play a role in metabolic dysfunction and systemic inflammation in human subjects. Excess subcutaneous fat leads to serious health issues which are associated with decreased life expectancy, such as type II diabetes and cardiovascular disease. In many cases, individuals desire to reduce subcutaneous fat and have difficultly doing so through diet and exercise alone.

Subcutaneous adipose tissue comprises adipocytes (fat cells) grouped together in lobules separated by connective tissues, and is not homogenous across all body areas. The size of adipocytes varies according to the nutritional state of the body, and the biology of adipocytes varies among different areas of the body. In some areas of the body, such as the torso, the subcutaneous adipose tissue is divided into two layers separated by a fascial plane, i.e., a superficial layer and a deep layer.

The superficial layer is called “superficial subcutaneous adipose tissue” (sSAT), and is bonded superiorly by the dermis and inferiorly by a fascial layer. The superficial layer is characterized by a lamellar pattern having regular, defined cuboid fat lobules tightly organized within vertically oriented fibrous septae. The superficial layer provides mechanical support and plays a thermos-insulator and metabolic role, and is highly vascularized.

The deep layer is called “deep subcutaneous adipose tissue” (dSAT), and is bounded superiorly by a fascial layer and inferiorly by muscle. The deep layer is different from the superficial layer in both form and function. The deep layer is characterized by a loose areolar pattern and has fat lobules which are flat shaped, irregular in size, and are surrounded by high amounts of loose connective tissue. The deep layer is less vascularized than the superficial layer, but is characterized by vessels of a larger lumen size. The deep layer plays a metabolic and inflammatory role in the body. dSAT is often the cause of poor aesthetic outcomes, such as excess adiposity. Both sSAT and dSAT layers also comprise sublayers each separated by a facial plane and compartments, separated by fibrous septae.

One method of treating humans suffering from medical and cosmetic issues related to excess fat is removal of the excess fat. Invasive fat reduction procedures include liposuction, abdominoplasty (“tummy tuck”), gluteoplasty (buttock lifts), brachioplasty (arm lift), thighplasty (thigh lift), lower rhytidectomy (neck lift), and mentoplasty (chin tightening). Invasive therapies carry risks associated with surgical procedures, some of which can be life threatening. These include infection, scarring, perforation of organs and vessels, and hemorrhage. Additionally, invasive therapies are often painful and typically require a lengthy recovery period.

Minimally invasive fat reduction procedures include laser-assisted liposuction, laser lipolysis (e.g., the breakdown of lipids), radio frequency lipolysis, ultrasound lipolysis, and injection lipolysis (e.g. injection of deoxycholic acid; KYBELLA). These procedures may require a surgical incision and/or the delivery of chemicals into the body, which can carry risks to the patient, and are often painful and produce non-uniform results.

dSAT is the main target of liposuction. Because dSAT has a loose density compared to the lamellar density of sSAT, dSAT is easier to remove using suction through a cannula. Removal of dSAT can allow for a more dramatic cosmetic and aesthetic improvement. Further, dSAT has an overlying layer of sSAT to mask the appearance of any irregularities and is more forgiving cosmetically than sSAT. Removal of fat has very little margin for error in areas of the body that have an sSAT layer but not a dSAT layer. Any minor irregularities in fat removal in the sSAT layer result in contour deformities in overlying skin, thereby contributing to a poor aesthetic result.

Noninvasive procedures to reduce undesirable subcutaneous fat include the use of radio frequency, lasers, ultrasound, and cryolipolysis. Cryolipolysis, or fat freezing, refers to cold-induced reduction of adipocytes. Given that lipid rich cells (such as subcutaneous fat and visceral fat) are more sensitive to cold injury than water-rich cells (such as skin and muscle), treatment of tissue with cool temperatures selectively targets fat cells and leaves other cell types unaffected. This concept of cryolipolysis has been used widely in devices that are placed on the skin to remove subcutaneous fat for aesthetic purposes. However, there are many limitations to topical cryolipolysis. Treatments are longer and colder than needed to selectively target fat, as the cold temperature needs to diffuse through the skin to the underlying subcutaneous fat. Further, topical cryolipolysis relies on an applicator which greatly limits the anatomic areas that can be treated (i.e., an area can only be treated if it can be accommodated by a standard applicator). Topical cryolipolysis also lacks precision, as the cold diffuses in an uncontrolled manner over a broad area during lengthy treatment times that are necessary for topical application. Because cooling of the fat can only be achieved by diffusion of cold through the skin to the subcutaneous fat, this greatly limits the depth and amount of fat that can be removed. Additionally, conventional non-invasive and minimally-invasive fat removal modalities, such as topical cryolipolysis and other energy-based therapies, such as topically applied laser, radiofrequency, and ultrasound, are limited by depth, and are only able to target sSAT.

### SUMMARY

The presence of excess fat causes a variety of health and cosmetic related concerns. Accordingly, there is a desire to provide methods for fat removal, in non-invasive manners. The present invention provides a composition comprising liquid water, and optionally one or more additives, wherein about 71% to about 100% of the water is in a solid, or frozen, state.

The present invention provides:

(1) A composition comprising liquid water, about 71% to about 100% by volume of water in a solid state, and optionally one or more additives.

(2) The composition according to the above (1), comprising about 95% to about 100% by volume of water in a solid state.

(3) The composition according to the above (1) or (2), comprising about 99% to about 100% by volume of water in a solid state.

(4) The composition according to any of the above (1) to (3), comprising at least one additive selected from the group consisting of a salt, a sugar and a thickener.

(5) The composition according to any of the above (1) to (4), comprising sodium chloride, glycerol and a thickener.

(6) A method for treatment of subcutaneous adipose tissue, the method comprising: administering the composition according to any of the above (1) to (5) to a treatment site of a subject, wherein the treatment site is selected from (i) superficial subcutaneous adipose tissue, (ii) deep subcutaneous adipose tissue, or (iii) superficial subcutaneous adipose tissue and deep subcutaneous adipose tissue.

(7) The method according to the above (6), wherein the composition is administered to superficial subcutaneous adipose tissue, followed by administration to deep subcutaneous adipose tissue.

(8) The method according to the above (6) or (7), wherein the composition is administered to deep subcutaneous adipose tissue, followed by administration to superficial subcutaneous adipose tissue.

(9) The method according to any of the above (6) to (8), wherein the composition is administered to deep subcutaneous adipose tissue and superficial subcutaneous adipose tissue simultaneously.

(10) The method of according to any of the above (6) to (9), wherein the composition is administered to a plurality of treatment sites.

(11) The method according to any of the above (6) to (10), wherein the composition is injected into the treatment site(s) of the subject.

(12) The method according to any of the above (6) to (11), wherein the composition is injected into the plurality of treatment sites, through a plurality of injection sites.

(13) The method according to any of the above (6) to (12), wherein the plurality of injection sites forms a pattern.

(14) The method according to any of the above (6) to (13), wherein the pattern is a plow pattern, a grid pattern, or a fan pattern.

(15) The method according to any of the above (6) to (14), wherein an incision is made to allow access to the treatment site, and the composition is administered by insertion at the treatment site of the subject through the incision.

(16) The method according to any of the above (6) to (15), wherein the composition is injected into the treatment site using a needle.

(17) The method according to any of the above (6) to (16), wherein the composition is injected into the plurality of treatment sites using one or more needles.

(18) The method according to any of the above (6) to (17), wherein the needle comprises a gauge size of about 8 G to about 25 G.

(19) The method according to any of the above (6) to (18), wherein the one or more needles comprise a gauge size of about 8 G to about 25 G, and the gauge size of each needle may be the same or different.

(20) The method according to any of the above (6) to (19), wherein the composition is injected into the treatment site using a cannula.

(21) The method according to any of the above (6) to (20), wherein the composition is injected into the plurality of treatment sites using one or more cannulas.

(22) The method according to any of the above (6) to (21), wherein the treatment of subcutaneous adipose tissue comprises initiating cryolipolysis.

(23) The method according to any of the above (6) to (22), wherein the treatment of subcutaneous adipose tissue further comprises tightening of the subcutaneous adipose tissue.

(24) The method according to any of the above (6) to (23), further comprising administering an anesthetic to the subject prior to and/or during the administration of the composition.

(25) A method of creating a treatment plan for a subject, said method comprising obtaining information from the subject prior to, during, and/or after administration of the composition of any of the above (1) to (5), providing the information to a computer or artificial intelligence system, and utilizing the computer or artificial intelligence system to create the treatment plan for the subject.

(26) The method according to the above (25), wherein the information obtained from the subject comprises one or more of gender, height, body weight, body fat percentage, septae rigidity, lifestyle, vitals, medical history, lipid profiles, skin elasticity, medication, nutrition, supplements, demographic, vascularity of fat tissue, and fat saturation.

(27) The method according the above (25) or (26), wherein the information is obtained from the subject using imaging.

(28) The method according to any of the above (6) to (23), wherein the method further comprises treatment of a condition, disorder or disease by administration of a therapeutically effective amount of the composition to the subject, wherein the condition, disorder or disease is one or more of excess fat, obesity, loose skin, metabolic dysfunction, insulin resistance, type II diabetes, systemic inflammation, inflammatory disorders, hypertension, hyperlipidemia, elevated triglycerides, dyslipidemia, fatty liver disease, obstructive sleep apnea, lipedema, lymphedema, non-alcoholic steatohepatitis, atrial fibrillation, atherosclerosis, systemic inflammation, inflammatory disorders, nerve pain, lipodystrophy, decrum's disease, lipomatosis, familial multiple lipomatosis, aberrant fat tissue proliferation, Proteus syndrome, Cowden Syndrome, Modeling disease (benign symmetric lipomatosis), familial angiolipomatosis, lymphatic leakage, de novo adipogenesis, increase in adipocyte cell size, and adipocyte proliferation due to excessive leakage of lymphatic including leakage of free fatty acids containing lymph, and wherein the subject is in need of treatment of the condition.

(29) A method for generating a composition comprising liquid water, about 71% to about 100% by volume of water in a solid state, and optionally one or more additives, said method comprising cooling a composition comprising liquid water and the optionally one or more additives until about 71% to about 100% by volume of water in a solid state is obtained.

(30) The method according to the above (29), wherein the cooling is achieved by freezing or supercooling the composition comprising liquid water and the optionally one or more additives.

(31) A method for generating a composition comprising liquid water, about 95% to about 100% by volume of water in a solid state, and optionally one or more additives, said method comprising cooling a composition comprising liquid water and the optionally one or more additives until about 95% to about 100% by volume of water in a solid state is obtained.

(32) The method according to any of the above (29) to (31), further comprising placing the composition comprising liquid water and the optionally one or more additives in a mold prior to freezing or supercooling.

(33) The method according to any of the above (29) to (32), wherein the mold is in a shape suitable for a desired treatment site.

(34) The method according to any of the above (29) to (33), wherein the mold shape is a cylinder, a cube, a cuboid, a sphere, a rounded mold, a triangular prism, a cone, a combination thereof, or a modification thereof.

(35) The method according to any of the above (29) to (34), wherein the mold shape is a unique shape.

(36) The method according to any of the above (29) to (35), wherein the mold comprises an open structure.

(37) The method according to any of the above (29) to (36), wherein the mold comprises a closed structure.

(38) The method according to any of the above (29) to (37), wherein the mold is a cannula or a needle.

(39) The method according to any of the above (29) to (38), wherein the mold has a selected size and a selected shape, wherein the selected size and the selected shape are determined through analysis of the treatment site.

(40) The method according to any of the above (29) to (39), wherein the analysis of the treatment site is conducted by measuring the treatment site, an area surrounding the treatment site, or a combination thereof.

(41) The method according to any of the above (29) to (40), wherein the measuring is conducted through visual measuring, measuring using imaging, computer assisted measuring, artificial intelligence assisted measuring, or a combination thereof.

(42) The method according to any of the above (29) to (41), wherein the mold is made of any suitable material.

### BRIEF DESCRIPTION OF THE DRAWINGS

FIG. 1 demonstrates the distribution of subcutaneous fat in males and females, and demonstrates mold shapes which may be suitable for specific locations in the body.

FIG. 2 is a diagram of an approach to generating a substantially solid solution inside a patient's body.

FIG. 3 is a view of a generating end of a point of delivery generation device for forming a substantially solid solution from liquid water and solid water.

FIG. 4A is a view of a generating end of another point of delivery generation device for forming a substantially solid solution from liquid water and solid water.

FIG. 4B is a sectional view of the generating end of FIG. 4A.

FIG. 5 is a view of a generating end of a point of delivery generation device for forming solid water from a first supply of liquid water and then forming a substantially solid solution from the solid water and a second supply of liquid water.

FIG. 6 is a view of a generating end of a point of delivery generation device for forming a substantially solid solution from supercooled water and ice pellets.

FIGS. 7A and 7B are views of a generating end of a point of delivery generation device for forming a substantially solid solution inside a balloon.

FIG. 8 is a view of a point of delivery generation device for generating and replenishing substantially solid solution.

FIG. 9 is a view of a point of delivery generation device having multiple working channels.

FIG. 10 shows small areas of the body suitable for discrete and targeted treatment.

FIG. 11 shows injection of a substantially solid solution into sSAT.

FIG. 12 shows injection of a substantially solid solution into dSAT.

FIG. 13 shows tissue rewarming of sSAT and dSAT in light of vascular supply.

FIG. 14 shows targeting and removal of sSAT.

FIG. 15 shows targeting and removal of dSAT.

FIG. 16 shows targeting and removal of sSAT and dSAT.

FIG. 17 shows active warming of the sSAT.

FIG. 18 shows active warming of the dSAT.

FIG. 19 shows variations in fat in the abdomen and lower thigh anatomic areas.

FIG. 20 shows variations in fat by the back and buttocks anatomic areas.

FIG. 21 shows an aspect of a system for generating a substantially solid solution.

### DETAILED DESCRIPTION

The invention provides substantially solid solutions, systems and methods for generating substantially solid solutions, and methods for administering substantially solid solutions to subjects for removal of fat in subcutaneous fat layers.

A substantially solid solution in accordance with the invention comprises water, and optionally one or more additives, wherein at least a portion of the water is in a frozen state, such that the ice coefficient (defined as the percentage of ice, i.e., the percent by volume of water in a solid state in the substantially solid solution, or the amount of ice by weight) may be in a range from about 71% to about 100%, including but not limited to 71%, 75%, 80%, 85%, 90%, 91%, 92%, 93%, 94%, 95%, 96%, 97%, 98%, 99%, 99.5%, 99.9% and 100%. A substantially solid solution, in accordance with the invention, enables high efficiency, by containing a large amount of ice at a low volume.

A substantially solid solution of the invention may be any suitable composition, comprising from about 71% to about 100% of water in a solid state, which is capable of removing adipocytes. In one aspect of the invention, the substantially solid solution may comprise 95 to 100%, including but not limited to 96%, 97%, 98%, 99%, 99.5% and 99.9%, of water in a solid state, e.g., an ice composition. In another aspect of the invention, the substantially solid solution comprising 95 to 100% of water in a solid state is an ice needle composition, wherein the ice needle composition may be generated in a mold which is a needle or a cannula. The substantially solid solution of the invention may be safe and effective for injection in humans. The substantially solid solution may comprise one or more additives, including but not limited to a freezing point depressant, a thickener, a surfactant, and a tonicity-modifying agent. In an aspect of the invention, the substantially solid solution comprises at least one of a freezing point depressant, and a thickener. In another aspect of the invention, the substantially solid solution comprises at least one of a salt, a sugar and a thickener.

A freezing point depressant lowers the freezing point of the substantially solid solution. Exemplary freezing point depressants include, but are not limited to, salts (e.g., sodium chloride, saline, potassium chloride, calcium chloride, magnesium chloride, sodium hydrogen phosphate or sodium carbonate), ions, Lactated Ringer's solution, sugars (e.g., glucose, sorbitol, mannitol, hetastarch, sucrose, or dextrose), biocompatible surfactants, glycerol, other polyols, other sugar alcohols, urea, and the like. In some aspects, the content of the freezing point depressant of the substantially solid solution is between about 0.5% and about 40%, between about 0.5% and about 30%, or between about 0.5% and about 5%.

Exemplary agents affecting the tonicity include, but are not limited to, salts, such as potassium chloride and sodium chloride, cations, anions, polyatomic cations, polyatomic anions, sugars such as dextrose, and sugar alcohols.

In some aspects, the one or more additives may comprise a buffer to stabilize the pH. In some aspects, the solution pH is about 4.5 to about 9. In some aspects, the one or more additives may comprise an emulsifier to create a smooth texture. In some aspects, the one or more additives may comprise a nanoparticle, for example, TiO₂. In some embodiments, the one or more additives may comprise an agent configured as coating for the water in a solid state, which may prevent agglomeration after formation. In some aspects, the one or more additives may comprise IVF Synthetic Colloids at amounts of about 6.0% Hetastarch in about 0.9% sodium chloride; Poloxamer 188 at amounts of about 0.2% subcutaneous; Propylene Glycol at amounts of about 0.47% to about 1.4%; Benzyl Alcohol at amounts of about 0.9% to about 1.4%; gelatin at amounts of about 16%; and Icodextrin used frequently in peritoneal dialysis at amounts of about 7.5%.

In some aspects, the one or more additives may comprise an excipient, such as those found in Sougata Pramanick et al., “Excipient Selection In Parenteral Formulation Development,” 45(3) Pharma Times 65-77 (2013), which is incorporated herein by reference. Exemplary excipients include, but are not limited to, bulking agents, such as sucrose, lactose, trehalose, mannitol, sorbitol, glucose, raffinose, glycine, histidine, PVP (K40); buffering agents, such as sodium citrate, sodium phosphate, sodium hydroxide, tris base-65, tris acetate, tris HCl-65; tonicity modifiers, such as dextrose; collapse temperature modifiers such as dextran, ficoll, gelatin, and hydroxyethyl starch; antimicrobial preservatives such as benzalkonium chloride, benzethonium chloride, benzyl alcohol, chlorobutanol, m-cresol, myristyl gamma-picolinium chloride, paraben methyl, paraben propyl, phenol, 2-phenoxyethanol, phenyl mercuric nitrate, and thimerosal; chelating agents such as calcium disodium EDTA (ethylenediaminetetraacetic acid), disodium EDTA, calcium versetamide sodium, calteridol, and DTPA; antioxidant and reducing agents such as acetone sodium bisulfate, argon, ascorbyl palmitate, ascorbate (sodium/acid), bisulfite sodium, butylated hydroxyl anisole, butylated hydroxyl toluene (BHT), cystein/cysteinate HCl, dithionite sodium, gentistic acid, gentistic acid ethanolamine, glutamate monosodium, glutathione, formaldehyde sulfoxylate sodium, metabisulfite potassium, metabisulfite sodium, methionine, monothioglycerol (thioglycerol), nitrogen, propyl gallate, sulfite sodium, tocopherol alpha, alpha tocopherol hydrogen succinate, thioglycolate sodium, thiourea, and anhydrous stannous chloride; solvents and co-solvents such as benzyl benzoate, oils, castor oil, cottonseed oil, N,N dimethylacetamide, ethanol, dehydrated ethanol, glycerin/glycerol, N-methyl-2-pyrrolidone, peanut oil, PEG, PEG 300, PEG 400, PEG 600, PEG 3350, PEG 4000, poppyseed oil, propylene glycol, safflower oil, sesame oil, soybean oil, vegetable oil, oleic acid, polyoxyethylene castor, sodium acetate—anhydrous, sodium carbonate—anhydrous, triethanolamine, and deoxycholate; buffers and pH-adjusting agents such as acetate, ammonium sulfate, ammonium hydroxide, arginine, aspartic acid, benzene sulfonic acid, benzoate sodium/acid, bicarbonate-sodium, boric acid/sodium, carbonate/sodium, carbon dioxide, citrate, diethanolamine, glucono delta lactone, glycine/glycine HCl, histidine/histidine HCl, hydrochloric acid, hydrobromic acid, lysine (L), maleic acid, meglumine, methanesulfonic acid, monoethanolamine, phosphate (acid, monobasic potassium, dibasic potassium, monobasic sodium, dibasic sodium and tribasic sodium), sodium hydroxide, succinate sodium/disodium, sulfuric acid, tartarate sodium/acid, and tromethamine (Tris); stabilizers such as aminoethyl sulfonic acid, asepsis sodium bicarbonate, L-cysteine, dietholamine, diethylenetriaminepentacetic acid, ferric chloride, albumin, hydrolyzed gelatin, insitol, and D,L-methionine; surfactants such as polyoxyethylene sorbitan monooleate (TWEEN® 80), Sorbitan monooleate, polyoxyethylene sorbitan monolaurate (TWEEN® 20), lecithin, polyoxyethylene-polyoxypropylene copolymers (PLURONICS®), polyoxyethylene monolaurate, phosphatidylcholines, glyceryl fatty acid esters, urea; complexing/dispersing agents such as cyclodextrins (e.g., hydroxypropyl-β-cyclodextrin, sulfobutylether-Bcyclodextrin); viscosity building agents such as sodium carboxymethyl cellulose (sodium CMC or CMC), xanthan gum, polyethylene glycol, acacia, gelatin, methyl cellulose, hydroxyethylcellulose, hydroxypropylmethylcellulose, methylcellulose, polyvinyl alcohol, polyvinylpyrrolidone, guar gum, locust bean gum, carrageenan, alginic acid, gelatin, acacia, and carbopol.

Any suitable concentration of one or more additives may be used in the substantially solid solution, and may be selected based upon the particular needs of the generation and/or administration of the substantially solid solution.

In some embodiments, the substantially solid solution may include water. In some embodiments, the substantially solid solution may include water and one or more additives. In some embodiments, the one or more additives are inactive, biocompatible ingredients, including any substance (at or below their respective indicated concentrations) in the FDA GRAS list, which is incorporated by reference in its entirety herein. In some embodiments, the additives can comprise one or more of a salt, a sugar, and a thickener.

In some aspects, the substantially solid solution comprises potassium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the substantially solid solution comprises calcium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the substantially solid solution comprises sodium chloride at about 2.25% by mass or lower, for example at about 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass. In some aspects, the substantially solid solution comprises magnesium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass.

In some aspects, the substantially solid solution comprises sucrose at about 5% by mass or lower, for example at about 5, 4.5, 4, 3.5, 3, 2.5, 2, 1.5, 1, 0.5, or 0% by mass. In some aspects, the substantially solid solution comprises dextrose at about 5.6% by mass or lower, for example at about 5.5, 5, 4.5, 4, 3.5, 3, 2.5, 2, 1.5, 1, 0.5, or 0% by mass. In some aspects, the substantially solid solution comprises mannitol at about 4.95% by mass or lower, for example at about 4.5, 4, 3.5, 3, 2.5, 2, 1.5, 1, 0.5, or 0% by mass. In some aspects, the substantially solid solution comprises lactose at about 0.45% by mass or lower, for example at about 0.4, 0.35, 0.3, 0.25, 0.2, 0.15, 0.1, 0.05, or 0% by mass. In some aspects, the substantially solid solution comprises sorbitol at about 4.7% by mass or lower, for example at about 4.5, 4, 3.5, 3, 2.5, 2, 1.5, 1, 0.5, or 0% by mass. In some aspects, the substantially solid solution comprises glycerol at about 2% by mass or lower, for example at about 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass.

In some aspects, the substantially solid solution comprises hetastarch at about 6% by mass or lower, for example at about 5.5, 5, 4.5, 4, 3.5, 3, 2.5, 2, 1.5, 1, 0.5, or 0% by mass. In some aspects, the substantially solid solution comprises pectin at about 16.7% by mass or lower, for example at about 16, 15, 10, 9, 8, 7, 6, 5, 4, 3, 2, 1 or 0% by mass. In some aspects, the substantially solid solution comprises polyethylene glycol at about 20% by mass or lower, for example at about 20, 15, 10, 9, 8, 7, 6, 5, 4, 3, 2, 1 or 0% by mass. In some aspects, the substantially solid solution comprises gelatin at about 16% by mass or lower, for example at about 15, 10, 9, 8, 7, 6, 5, 4, 3, 2, 1 or 0% by mass. In some aspects, the substantially solid solution comprises sodium methylcellulose at about 5% by mass or lower, for example at about 5, 4.5, 4, 3.5, 3, 2.5, 2, 1.5, 1, 0.5, or 0% by mass. In some aspects, the substantially solid solution comprises a sodium alginate at about 5% by mass or lower, for example at about 5, 4.5, 4, 3.5, 3, 2.5, 2, 1.5, 1, 0.5, or 0% by mass. In some aspects, the substantially solid solution comprises polyvinyl alcohol at about 5% by mass or lower, for example at about 5, 4.5, 4, 3.5, 3, 2.5, 2, 1.5, 1, 0.5, or 0% by mass. In some aspects, the substantially solid solution comprises polyvinyl pyrrolidone (PVP) at about 5% by mass or lower, for example at about 5, 4.5, 4, 3.5, 3, 2.5, 2, 1.5, 1, 0.5, or 0% by mass. In some aspects, the substantially solid solution comprises Xanthan Gum at about 0.75% by mass or lower, for example at about 0.7, 0.6, 0.5, 0.4, 0.3, 0.2, 0.1, 0.05 or 0% by mass. In some aspects, the substantially solid solution comprises CMC at about 0.75% by mass or lower, for example at about 0.7, 0.6, 0.5, 0.4, 0.3, 0.2, 0.1, 0.05 or 0% by mass. In some aspects, the substantially solid solution comprises guar gum at about 1% by mass or lower, for example at about 0.9, 0.8, 0.7, 0.6, 0.5, 0.4, 0.3, 0.2, 0.1, 0.05 or 0% by mass. In some aspects, the substantially solid solution comprises locust bean gum at about 1% by mass or lower, for example at about 0.9, 0.8, 0.7, 0.6, 0.5, 0.4, 0.3, 0.2, 0.1, 0.05 or 0% by mass. In some aspects, the substantially solid solution comprises gum tracanth at about 1% by mass or lower, for example at about 0.9, 0.8, 0.7, 0.6, 0.5, 0.4, 0.3, 0.2, 0.1, 0.05 or 0% by mass. In some aspects, the substantially solid solution comprises carbomer at about 1% by mass or lower, for example at about 0.9, 0.8, 0.7, 0.6, 0.5, 0.4, 0.3, 0.2, 0.1, 0.05 or 0% by mass.

In some aspects of the invention, for intradermal, subcutaneous, or intramuscular routes of administration, additives may include sodium chloride, saline, glycerin/glycerol, dextrose, sodium CMC, xanthan gum, and polyethylene glycol. For example, acceptable concentrations of saline are about 0.9% for soft tissue use and about 2.25% for subcutaneous use, while acceptable concentrations of glycerin/glycerol are about 1.6% to about 2.0% for dermal use and about 15% for subcutaneous use. For example, acceptable concentrations of dextrose are about 5% w/v for intramuscular use and about 7.5% per unit dose for intramuscular-subcutaneous use. For example, acceptable concentrations of sodium CMC are about 0.75% for intralesional use, about 3% for intramuscular use, and about 0.5% to about 0.75% for soft tissue use. As another example, acceptable concentrations of xanthan gum are about 1% for intra-articular use in animal studies and about 0.6% for FDA ophthalmic use. For example, acceptable concentrations of polyethylene glycol, such as Polyethylene Glycol 3350, are about 2.0% to about 3.0% for FDA soft tissue use and about 4.42% for subcutaneous use.

In certain aspects, the substantially solid solution has an osmolarity lower than about 2,200 mOsm/L. In some aspects, the osmolarity is less than about 1,000 mOsm/L. In some aspects, the osmolarity is less than about 600 mOsm/L. In such an aspect, the substantially solid solution may comprise about 0.9% saline; about 1.0% to about 2.0% dextrose; about 1.0% to about 1.6% glycerol; less than about 0.5% sodium CMC; and less than about 0.6% xanthan gum. In one aspect, the substantially solid solution may be about 500 mOsm/kg to about 700 mOsm/kg and comprise about 0.9% to about 1.4% saline; about 2.0% to about 4.0% dextrose; about 1.7% to about 2.0% glycerol; about 0.6% to about 1.0% sodium CMC; and about 0.6% to about 1.0% xanthan gum. In another aspect, the substantially solid solution composition may be about 700 mOsm/kg to about 900 mOsm/kg and comprise about 1.5% to about 1.7% saline; about 5.0% to about 7.5% dextrose; about 3.0% to about 5.0% glycerol; about 1.0% to about 3.0% sodium CMC; and about 1.0% xanthan gum. In some aspects, the substantially solid solution composition may be greater than about 1,000 mOsm/kg. In such an aspect, the substantially solid solution may comprise about 1.8% to about 3.0% saline; about 10% dextrose; greater than about 5.0% glycerol; sodium CMC; and xanthan gum. In another aspect, the substantially solid solution can be isotonic relative to the subject's cells, e.g., having an osmolarity of about 308 mOsm/kg. In such an aspect, the substantially solid solution may include normal saline and 2% glycerol. Additives can be selected and included in any concentration suitable to generate a substantially solid solution have certain characteristics, for example to increase or decrease the osmolality.

The substantially solid solution of the present invention may be generated by any acceptable system and/or method which will provide the desired substantially solid solution.

Methods and systems described in International Application No. PCT/US19/55633, which is incorporated herein by reference, may be utilized to generate a substantially solid solution of the present invention, wherein the ice coefficient is about 71% to about 100%.

In an aspect of the invention, a system is provided for generating a substantially solid solution having an ice coefficient of about 95 to about 100%, including but not limited to 96%, 97%, 98%, 99%, 99.5% and 99.9%, for example, an ice composition. In some aspects, a solution comprising water and optionally one or more additives is frozen or supercooled in a mold or a delivery device, until the desired ice coefficient is reached. In some aspects, a substantially solid solution may be passively or actively melted, to obtain a substantially solid solution with a lower ice coefficient.

In an aspect of the invention, a system is provided for generating a substantially solid solution in the form of an ice needle. In an ice needle system, a solution comprising water and optionally one or more additives is frozen or supercooled in a cylindrical mold, including but not limited to tubing, a cannula or a needle, until an ice coefficient of about 95% to about 100%, including but not limited to 96%, 97%, 98%, 99%, 99.5% and 99.9%, is obtained. In this system, the resulting ice composition may comprise a cylindrical shape, such as a needle, i.e., an ice needle.

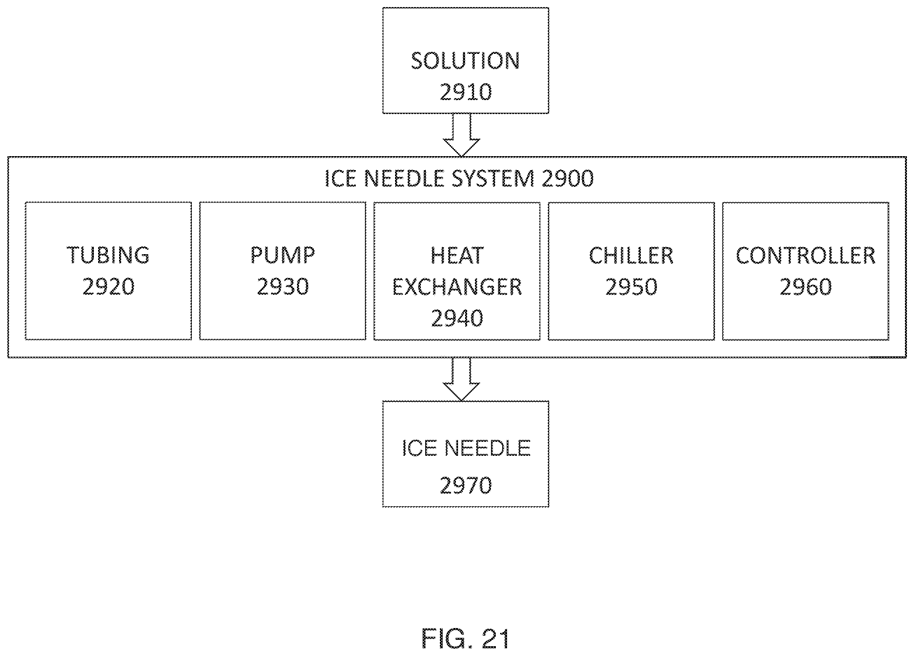

FIG. 21 shows a non-limiting example of an ice needle system 2900 for generating an ice needle 2970. The inlet and outlet may be any suitable container, such as a solution container. The solution 2910 is pumped through tubing 2920 by a pump 2930, which can be any suitable pump such as an HPLC Pump. Any suitable tubing may be used. In some aspects, the tubing has a narrow inner diameter, for example, comparable to a syringe needle such as an 8-25 G needle such that sufficient flowability is achieved. The tubing may comprise a slight curvature (or other suitable configuration) to break up the ice as it is dispensed. In some embodiments, the solution is cooled using a heat exchanger 2940 and chiller 2540. A controller 2960 controls parameters of the pump 2930, heat exchanger 2940, and chiller 2950, such as flowrate (e.g., 10 ml/min), pressure, and temperature. The controller 2960 communicates with the system components but may be internal to the system or external to the system.

In an aspect of the invention, a point of delivery system is provided for generating a substantially solid solution at the point of delivery to the subject. In such an aspect, water in a solid state and a solution comprising liquid water and optionally one or more additives are combined in a delivery device, such as a needle or a cannula, such that the liquid water and optionally one or more additives and the solid water mix together as they exit the delivery device in order to form a substantially solid solution.

A substantially solid solution having an ice coefficient of about 95 to 100%, i.e., an ice composition, may be generated through the use of a mold. Utilizing a mold when generating the ice composition allows the ice to take the desired shape and size, e.g., a shape and size which are appropriate for the area of the body to be treated, e.g., the treatment site. A treatment may include more than one treatment site, and may require selection of more than one mold.

The shape of the mold may be any shape suitable for the treatment site, including but not limited to cylinders, such as tubing, a cannula or a needle, cubes, cuboids, spheres, rounded molds, triangular prisms, cones, combinations thereof, and modifications thereof. Any of these shapes may also include a camber, or slight convexity, arching, or curvature, dependent on the shape of the treatment site. The shape of the mold may also be a unique shape which is formed after analysis of the desired treatment site. The mold may comprise a closed structure, wherein the mold is entirely enclosed but for an opening from the inside to the outside of the mold, or an open structure, wherein the mold is open on one side. The size of the mold may be any size suitable for the treatment site.

In an aspect of the invention, when a cylindrical mold is employed, the mold may have a pointed end, to aid with insertion into the treatment site. In another aspect of the invention, when a cylindrical mold is employed, the mold may comprise a slight curvature at the end (or other suitable configuration), to aid in breakage of the ice as it is dispensed from the mold, for example, when an ice needle is administered directly to the treatment site from the mold. In another aspect of the invention, the mold may contain a coil, which allows the formation of individual units of the substantially solid solution.

In selecting the appropriate mold, the treatment site should be analyzed to determine the appropriate mold shape and size for treatment. In one aspect, the shape and size of the mold may be determined through visual analysis of the area of the treatment site. For example, when the treatment site is a small area, e.g., the chin of a patient, upon visual analysis of the treatment site, a needle shaped mold may be selected due to the small size of the treatment site. In a second aspect, the shape and size of the mold may be determined through a more detailed analysis of the treatment site, optionally in combination with a visual analysis.

In the second aspect, measurements of the treatment site may be necessary. Measurements of the treatment site and/or the surrounding area may be obtained by forming an incision near or at the treatment site to provide access to the desired treatment site, followed by taking measurements of the treatment site and/or the surrounding area. Such measurements provide guidance in determining the specific dimensions of the appropriate mold.

In the second aspect, measurements of the treatment site and/or the surrounding area may be obtained through imaging, including but not limited to Magnetic Resonance Imaging (MRI), Computed Topography (CT), ultrasound, Positron Emission Tomography (PET), 3D imaging, and combinations thereof. The results of such imaging may be studied to determine the specific dimensions of the appropriate mold. Imaging may be used in the absence of, or together with, an incision near or at the treatment site.

In the second aspect, measurements of the treatment site and/or the surrounding area may be obtained through a computer or artificial intelligence system, which contains data obtained from the subject to be treated and/or data obtained from multiple subjects.

By selecting a mold of a suitable size, high ice coefficients may be achieved through small ice needles. If an ice coefficient of 100% is desired, the ice is fully frozen within the mold. To generate a substantially solid solution having a high ice coefficient, for example an ice needle of 99 to 100%, generating the ice needle in a mold will alleviate concerns regarding pauses in the flow blocking the tubing.

After determining the desired mold shape and size, a mold can be obtained. The mold may be an existing device which satisfies the desired mold shape and size requirements, a prefabricated mold, or a mold created through any known method, including but not limited to 3D printing.

In an aspect of the invention, a cylindrical mold may be a cannula or a needle. The size of the needle or cannula may vary, and may be selected based upon the dimensions of the treatment site. The size of the needle or cannula includes but is not limited to about 8 G to about 25 G.

The mold may be formed from any suitable material which allows the substantially solid solution to be formed through supercooling or freezing, and which allows removal of the substantially solid solution for administration, or administration of the substantially solid solution directly from the mold. In one aspect of the invention, the mold comprises a material that acts as a temperature barrier which separates the mold material and the substantially solid solution. In other aspect, the mold comprises a biocompatible and/or biodegradable material, which may be administered together with the substantially solid solution and protects non-targeted tissue. After administration of the substantially solid solution, the biocompatible and/or biodegradable material is removed from the treatment site, or is absorbed into the treatment site.

After selecting and obtaining the desired mold, a solution comprising water and optionally one or more additives, is placed into the mold, followed by cooling the solution until the desired ice coefficient is achieved. The solution comprising water and optionally one or more additives may be placed into the mold in any suitable manner. Cooling the solution may be performed by any suitable method, including freezing or supercooling the mold containing the solution. Suitable methods for freezing or supercooling the substantially solid solution are disclosed in International Application No. PCT/US19/55633, which was previously incorporated herein by reference.

Supercooling is the process of lowering the temperature of the solution below its freezing point. As the solution is cooling down in a substantially solid solution generation system, a constant cooling temperature is maintained, and upon the solution reaching a temperature, nucleation can be initiated. In some embodiments, the temperature of the solution is cooled to or below about 10° C., 7° C., 5° C., 4° C., 3° C., 2° C., 1° C., 0° C., −1° C., −2° C., −3° C., −4° C., −5° C., −10° C., −15° C., −20° C., −30° C., −40° C., and −50° C. Temperature control can help to avoid formation of ice particles on smooth surfaces of the system, e.g., the mold.

The ice coefficient can be determined through weight analysis. For example, the sum of the weight of the solution and the mold is determined through any suitable measuring method. At desired stages during the cooling process, the weight of the mold containing the solution, which is transforming into a substantially solid solution, is obtained. Calculations are performed to determine the desired weight of a mold containing a substantially solid solution which correlates to the desired ice coefficient.

In one aspect, the method for forming the substantially solid solution may include cooling the solution comprising water and optionally one or more additives, in the selected mold, until the desired ice coefficient is achieved. In another aspect, the method for forming the substantially solid solution may include cooling the solution comprising water and optionally one or more additives, in the selected mold, until a substantially solid solution having an ice coefficient which is higher than the desired ice coefficient is achieved, and then allowing the substantially solid solution to melt until the desired ice coefficient is achieved. The melting may be passive, e.g., allowing the mold and substantially solid solution to melt at room temperature, or may be active, e.g., warming the mold and substantially solid solution to obtain the desired ice coefficient. The warming may be performed by any suitable method.

After generating the substantially solid solution, for example an ice composition such as an ice needle, the substantially solid solution may be removed from the mold for administration to the treatment site, or may be administered directly to the treatment site from the mold, or in combination with the mold.

In an aspect of the invention, the substantially solid solution may be pushed out of the mold directly into the desired treatment site. In some aspects, a substantially solid solution having an ice coefficient of about 95 to about 100% is formed using a cylindrical mold, such as a cannula or needle, thus generating an ice composition having a needle shape, e.g., an ice needle composition. In some aspects, when a cylindrical mold is employed, the mold may have a pointed end, thus resulting in an ice needle composition with a pointed tip for easier insertion directly into the desired treatment site. In some aspects, when a cylindrical mold is employed, the mold may curve at the end, to aid in breakage of the ice prior to exiting the mold. Such a feature may be used independently, or in combination with a mold with a pointed tip, and is useful when administering an ice needle directly from the mold to the treatment site. Where appropriate or desired, the mold may be warmed prior to pushing the ice composition out of the mold, in order to improve discharge of the ice composition from the mold.

In some aspects, the substantially solid solution is generated in a mold in a manner previously disclosed, followed by removal of the substantially solid solution, including an ice composition, from the mold, and transferal of the substantially solid solution to a delivery device for administration to the treatment site. The delivery device may be at a temperature lower than the temperature of the substantially solid solution, including but not limited to room temperature.

Any suitable method for removing the substantially solid solution, including an ice composition, from the mold may be utilized. In one aspect, a flexible mold material may be utilized, such that the mold may be physically manipulated after freezing or supercooling in order to release the substantially solid solution from the mold. In another aspect, the mold may be warmed, or briefly submerged in warm water, thus melting a portion of the substantially solid solution and allowing for ease in removal from the mold.

In another aspect, the mold and/or substantially solid solution may be coated with a material prior to adding the solution comprising water and optionally one or more additives, to aid in removal of the substantially solid solution from the mold. The coating material may be any biocompatible material which has a freezing point lower than the freezing point of the solution to be frozen or supercooled, including but not limited to, a biocompatible hydrophobic material such as glycerol, a slurry have an ice coefficient of 70% or less (slurry described in detail below), or a freezing point depressant.

When the mold is a cannula or needle, the cannula or needle may be connected directly to a delivery device, including but not limited to a syringe and pump, and delivered to the treatment site. The size of the needle or cannula may vary, and may be selected based upon the dimensions of the treatment site. The size of the needle or cannula includes but is not limited to 8 G to 25 G. The mold and/or delivery device may be warmed, using any suitable method, in order to enable transfer of the substantially solid solution from the mold to the delivery device.

The delivery device and/or the substantially solid solution may be coated with a material, prior to transferring the substantially solid solution to the delivery device, in order to aid in delivery of the substantially solid solution to the treatment site. The coating material may be any suitable material, including silicone fluid lubricants, diamond-like carbon coatings, metallic glass, thin film metallic glass, and the like, in order to reduce friction and allow ease of administration from the delivery device to the treatment site.

When the mold contains a coil, or a container having multiple chambers, individual units of the substantially solid solution may be formed. The coil or container may be made of any suitable material. For delivery of the individual units of the substantially solid solution, the coil comprising individual units of the substantially solid solution is removed from the mold via any suitable method, and then inserted into an appropriate delivery device, including but not limited to a device containing an injection portion and a trigger mechanism, allowing the individual units of the substantially solid solution to be administered to the desired treatment site. In this aspect, one or more individual units of the substantially solid solution are administered to the treatment site by engaging the trigger mechanism.

In an aspect of the invention, a sterile bag is obtained and a solution comprising water and optionally one or more additives is placed in the sterile bag, which is placed in a mold. In some aspects, the treatment specialist, including persons preparing the substantially solid solutions and persons administering the substantially solid solution, may prepare the solution comprising water and optionally one or more additives, and place the solution in the sterile bag. In other aspects, the treatment specialist preparing the system may obtain previously prepared sterile bag comprising a solution comprising water and optionally one more additives. In each aspect, the treatment specialist places the sterile bag comprising the solution comprising water and optionally one or more additives into a selected mold, and cools the mold, sterile bag and solution until the desired ice coefficient is achieved.

In an aspect of the invention, the substantially solid solution may be delivered to the treatment site via incision. After obtaining the substantially solid solution, for example an ice composition, an incision is made at or near the treatment site, and the substantially solid solution is delivered directly to the treatment side. In another aspect, an incision is made in a location of the body which is hidden from general observation, and a large needle is inserted into the incision site. For example, a 12 G needle may administer a 6 inch long needle shaped ice composition, thus providing about 3.5 cc of substantially solid solution to the desired treatment site. Delivery through one or more incisions allows flexibility in the mold shape and size.

In another aspect of the invention, the substantially solid solution may be delivered to the treatment site via injection. The injection may be administered using any suitable delivery device, including but not limited to, a syringe, a needle or a cannula. In some aspects, small portions of a substantially solid solution, may be administered in a repetitive manner through a suitable delivery device, including but not limited to, a cannula. In this aspect, the substantially solid solution is not administered as a single portion, but rather as multiple discrete portions, including but not limited to cylinders, rods, or spheres of the substantially solid solution. The discrete portions may be formed by barrier materials in the mold, or may be formed through manipulation of the substantially solid solution after cooling, including but not limited to mechanical manipulation.

In another aspect of the invention, the substantially solid solution may be administered through utilization of a guide device, including but not limited to, a guidewire. A guide device may be used to navigate small treatment areas, providing tactile feedback to the administrator of the treatment, which can be used to adjust directional movement. Further, a guide device, once properly placed, allows an additional, larger and less controllable device to more easily enter and arrive at the desired treatment site.

The guide device may be made of any suitable material which is appropriate for insertion into the treatment site, including but not limited to stainless steel. Guide devices, such as guidewires, may differ in material, presence or absence of a coating, size, e.g., diameter and length, shape and style of the tip, flexibility, maneuverability, and strength. The appropriate guide device should be selected based upon the particular needs of the treatment.

Additionally, utilization of a guidewire may prevent solids in the ice composition from breaking prior to placement at the treatment site.

In an aspect of the invention, the substantially solid solution may be formed around a guide device. For example, a guide device may be inserted into a cylindrical mold, such as a needle or cannula, followed by placing a solution comprising water and optionally one or more additives into the mold containing the guide device. After cooling the solution comprising water and optionally one or more additives to obtain a substantially solid solution having the desired ice coefficient, the mold is removed in a suitable method described above. The substantially solid solution, together with the encompassed guide device, are administered to the treatment site through any suitable method. For example, the substantially solid solution, with the guide device enclosed, may be administered via injection through a needle. In some aspects, the guide device may remain until treatment is complete, or may be removed after placement of the substantially solid solution in the desired treatment site. In some aspects, the guide device may be heated in order to ease retraction. In another aspect of the invention, the guide device may be utilized to break the solid portions of the substantially solid solution, for example, through mechanical manipulation or vibration of the guide device.

In an aspect of the invention, a catheter may be placed at or near the treatment site, thus allowing for multiple administrations during a single treatment session, or during multiple treatment sessions over one or more days.

In an aspect of the invention, a sterile bag is filled with a solution comprising water and optionally one or additives, and the sterile bag comprising the solution is administered to the treatment site by any suitable method, including but not limited to administration via a catheter. The sterile bag may have a size and shape suitable for the treatment site, including but not limited to a spherical shape or a cube shape. Upon administration, the sterile bag and solution are cooled to obtain a substantially solid solution having the desired ice coefficient, followed by removal of the sterile bag by any suitable method, thereby allowing the substantially solid solution to remain at the treatment site. The remaining substantially solid solution would take the shape of the bag, for example, a spherical shape or a cube shape, such as an ice cube.

In an aspect of the invention, the substantially solid solution may be generated and delivered at the treatment site, or the point of delivery. The substantially solid solution may be made in situ at the point of delivery of a subject. The components used to generate the substantially solid solution are provided under conditions that result in the formation of a substantially solid solution having the desired ice coefficient for a desired treatment protocol.

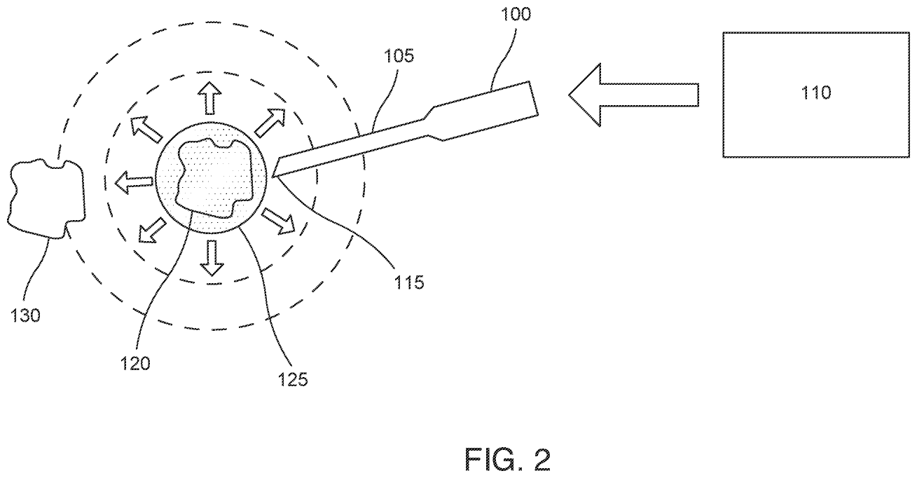

FIG. 2 shows an example of a point of delivery generation device 100 for making a substantially solid solution inside a subject's body. The device 100 includes an application cannula 105 having a shape and size configured to be inserted through a subject's skin. The device 100 is fluidly coupled to a supply 110 providing components for making a substantially solid solution. At the distal end of the application cannula 105, there is a generating end 115 for forming a substantially solid solution from the components.

The point of delivery generation device 100 is used by inserting the application cannula 105 through the subject's skin and advancing the generating end 115 to a location at or near a target tissue or treatment site 120 (shown in phantom line). The target tissue 120 can, for example be subcutaneous adipose tissue. The solution ingredients, such as water and optionally one or more additives, are pumped or otherwise conveyed, separately, from the supply 110, through the application cannula 105, and out the generating end 115. At the generating end 115, the components interact with each other and form the substantially solid solution 125 at or near the target tissue 120.

The cooling effect of the substantially solid solution 125 is localized to the target tissue 120 and possibly surrounding tissue, such as adjacent tissue 130. In this way, discomfort caused by the treatment is limited. The substantially solid solution is sterile and biocompatible; and, as such, the substantially solid solution 125 can be advantageously left in the body (e.g. no removal of the substantially solid solution is necessary after cooling has been effected).

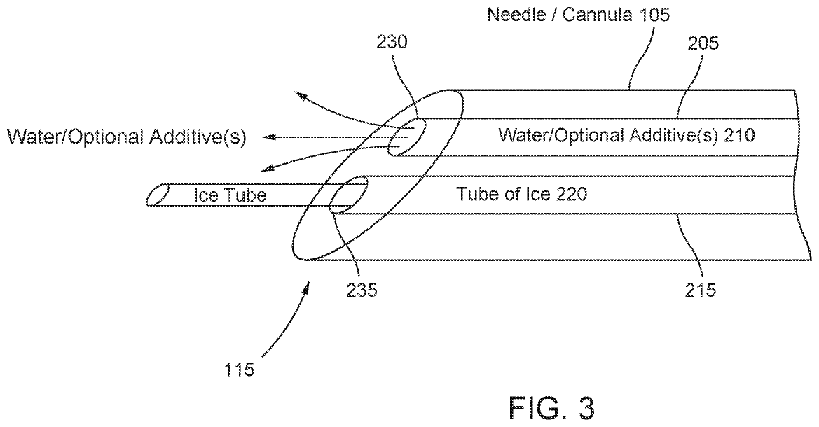

FIG. 3 shows an example of the generating end 115 for making a substantially solid solution from mixing a solution comprising water and optionally one or more additives and water in a solid state, i.e., solid water. The cannula 105 houses a first delivery cannula 205 for supplying liquid water and optionally one or more additives 210 and a second delivery cannula 215 for supplying solid water (ice) 220. The distal end of the first delivery cannula 205 is open and forms a first outlet 230 for the liquid water and optionally one or more additives 210 to exit. The distal end of the second delivery cannula 215 is open and forms a second outlet 235 for the solid water 220 to exit. The outlets 230, 235 are arranged so that the liquid water and optionally one or more additives 210 and the solid water 220 mix together as they exit to form a substantially solid solution. In some aspects, the second delivery cannula 215 can be configured to supply a substantially solid solution including ice and one or more additives as described herein.

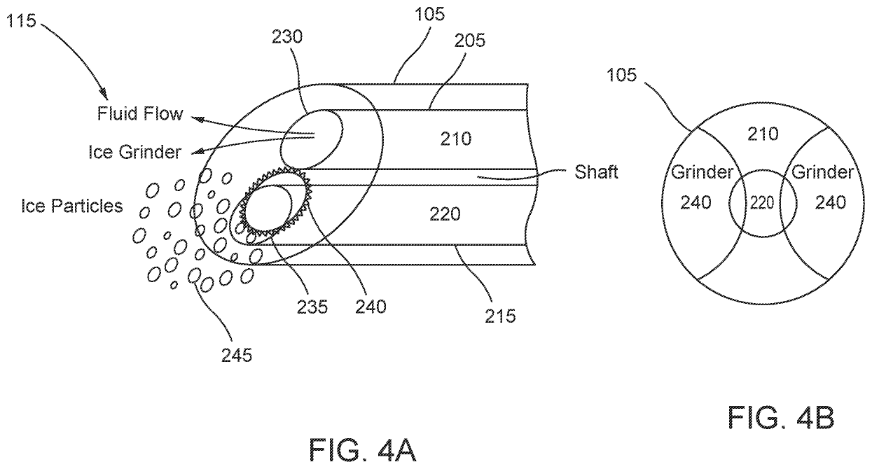

FIG. 4A shows an example of the generating end 115 for making a substantially solid solution from mixing liquid water and optionally one or more additives, and solid water. This example is similar to the one described above with reference to FIG. 3 with the addition of a grinder 240 located in front of the second outlet 235. The arrangement of the grinder 240 with respect to the second outlet 235 is better seen in the cross-sectional view of FIG. 4B. As the solid water 220 emerges from the second delivery cannula 215, the grinder 240 breaks the solid water 220 into particles 245. The solution comprising liquid water and optionally one or more additives 210 exiting from the first delivery cannula 205 mixes with the particles 245 to form a substantially solid solution. In another example (not shown), a vibrator can break the solid water into particles to combine with the solution comprising water and optionally one or more additives to form a substantially solid solution at the point of delivery.

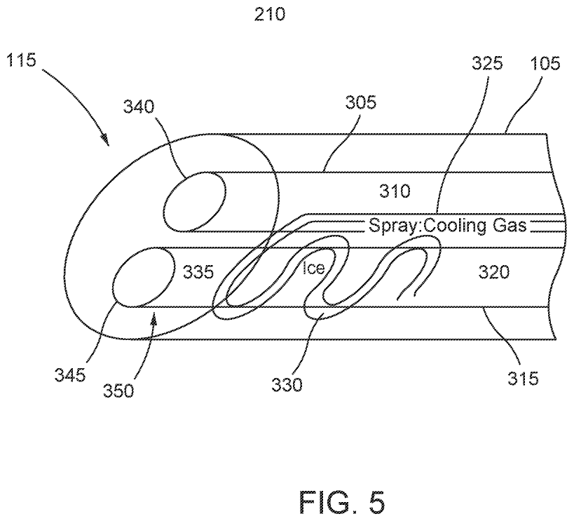

FIG. 5 shows another example of the generating end 115 for making a substantially solid solution from mixing liquid water and optionally one or more additives, and solid water. The application cannula 105 houses a first delivery cannula 305 for providing a first supply of liquid water and optionally one or more additives 310, and a second delivery cannula 315 for providing a second supply of liquid water 320. As shown, the application cannula 105 further includes a gas line 325 for spraying a cooling gas 330 and freezing the second supply of water 320 into solid water 335.

The distal end of the first delivery cannula 305 is open forming a first outlet 340 for the first supply of liquid water and optionally one or more additives 310 to exit. The distal end of the second delivery cannula 315 is open forming a second outlet 345 for the solid water 335 to exit. In front of the second outlet 345, there is a grinder (or vibrator) 350 to break the solid water 335 into particles as it emerges from the second delivery cannula 315. The outlets 340, 345 are arranged so that the first supply of liquid water and optionally one or more additives 310 and the particles of solid water mix together to form a substantially solid solution.

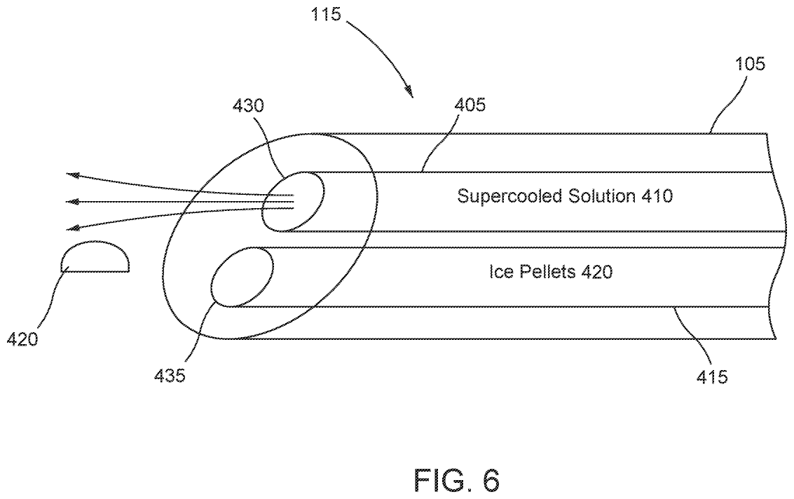

FIG. 6 shows an example of the generating end 115 for making a substantially solid solution from crystalizing a supercooled solution comprising water and optionally one or more additives. The application cannula 105 houses a first delivery cannula 405 for supplying a supercooled solution comprising water and optionally one or more additives 410. Water normally freezes at 273.15 K (0° C. or 32° F.), but it can be “supercooled” at standard pressure down to its crystal homogeneous nucleation at almost 224.8 K (−48.3° C./−55° F.). The freezing point of the solution may vary depending upon the presence of one or more additives. The supercooling process requires that water be pure and free of nucleation sites. This can be done by processes like reverse osmosis or chemical demineralization. Rapidly cooling water at a rate on the order of 10⁶ K/s avoids crystal nucleation and water becomes a glass, i.e., an amorphous (non-crystalline) solid.

The application cannula 105 further houses a second delivery cannula 415 for supplying ice pellets 420, which serves as nucleation sites for the crystallization process. The distal end of the first delivery cannula 405 is open and forms a first outlet 430 for the supercooled water 410 to exit. The distal end of the second delivery cannula 415 is open and forms a second outlet 435 for the ice pellets 420 to exit. The outlets 430, 435 are arranged so that the supercooled water 410 interacts with the ice pellets 420 causing it to crystalize and form a substantially solid solution.

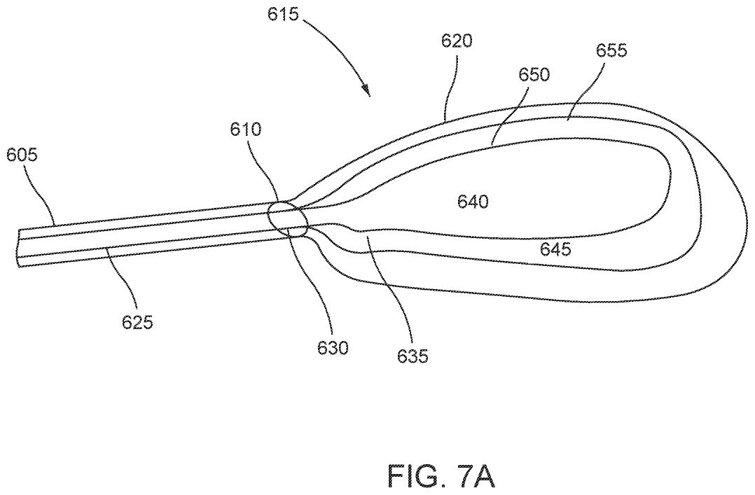

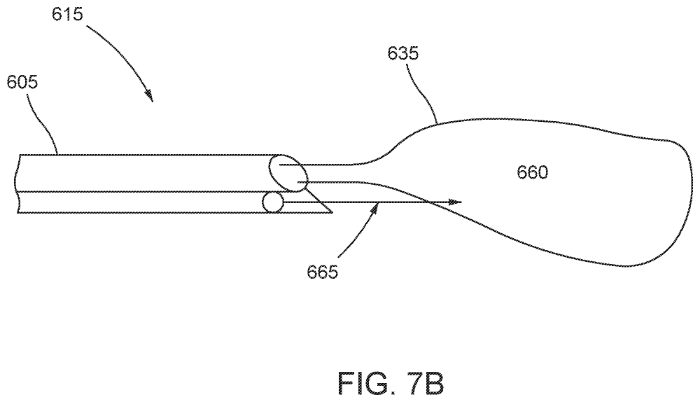

FIG. 7A shows another example of the point of delivery generation device. The device includes an application cannula 605 that is open at its distal end defining an outlet 610. A generating end 615 includes an outer balloon 620 disposed around the outlet 610. The application cannula 605 is in fluid communication with the interior volume of the outer balloon 620. The application cannula 605 includes a fluid delivery cannula 625. The application cannula 605 and the fluid delivery cannula 625 share a common longitudinal axis and can be said to be coaxially aligned.

The fluid delivery cannula 625 is open at its distal end defining a fluid outlet 630. The generating end 615 further includes an inner balloon 635 disposed around the fluid outlet 630. The fluid delivery cannula 625 is in fluid communication with an interior volume of the inner balloon 635, which is labeled 640 in the figure. The inner balloon 635 is located inside the outer balloon 620. As shown, the inner balloon 635 occupies a portion of the interior volume of the outer balloon 620 leaving a space or gap 645 between an outer wall of the inner balloon 635 (which is labeled 650 in the figure) and an inner wall of the outer balloon 620 (which is labeled 655 in the figure).

To generate a substantially solid solution at the point of delivery, the application cannula 605 is inserted through a subject's skin and the generating end 615 is advanced to a location at or near a target tissue in much the same manner as described above with reference to FIG. 2. In this example, the outer balloon 620 and the inner balloon 635 are inserted into the patient's body in their uninflated state. The inner balloon 635 is then filled or inflated with a cool solution comprising cold water and optionally one or more additives that is supplied through the fluid delivery cannula 625.

Once the inner balloon 635 is filled with the cool solution, the outer balloon 620 is filled with a cooling gas or fluid, such as liquid nitrogen. The cooling gas fills the gap 645 between the inner balloon 635 and the outer balloon 620. This causes the cool solution in the inner balloon 635 to partially freeze and form a substantially solid solution 660, as shown in FIG. 7B. (For clarity the outer balloon 620 is not shown in FIG. 7B.) The substantially solid solution-filled inner balloon 635 can then be used to cool a target tissue. Alternatively, the inner balloon 635 can be ruptured by a retractable puncture needle 665 that extends beyond the application cannula 605 when extended as shown. Rupturing the inner balloon 635 releases the substantially solid solution 660 at or near the target tissue.

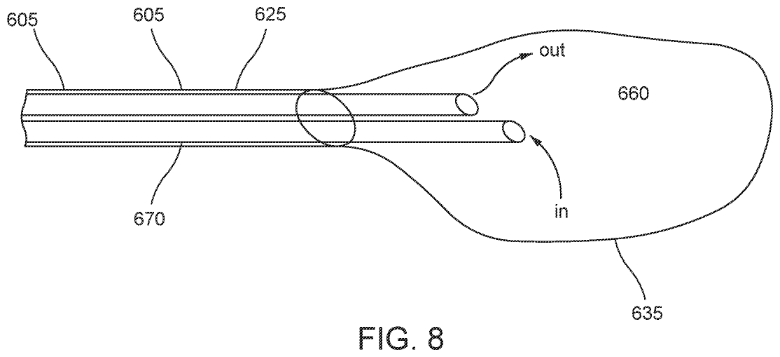

FIG. 8 shows another example of the point of delivery generation device for generating and replenishing a substantially solid solution. This example is similar to the one described above with reference to FIG. 7 with the addition of a fluid return cannula 670. (For clarity the outer balloon 620 is not shown in FIG. 8.) The fluid return cannula 670 is housed within the application cannula 605 together with the fluid delivery cannula 625, as shown. The fluid return cannula 670 removes substantially solid solution from the inner balloon 635 that is no longer at the desired temperature. Replenishing the “old” substantially solid solution with “fresh” substantially solid solution in this manner can accommodate for the eventually melting of substantially solid solution. In some aspects, a treatment specialist can administer a substantially solid solution having a higher ice coefficient, extending the period of cooling. This approach is particularly useful for a treatment requiring a long period of cooling.

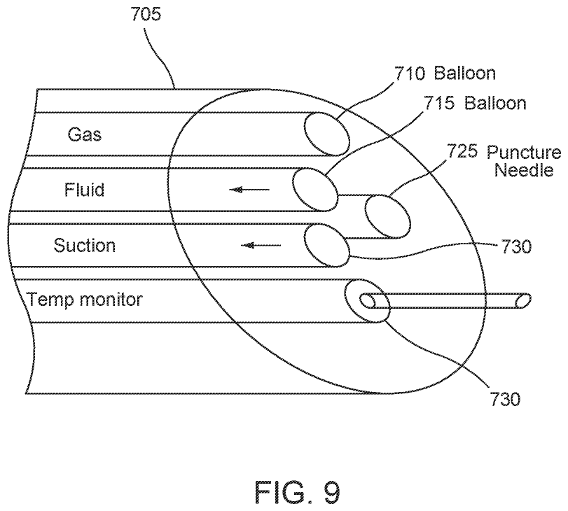

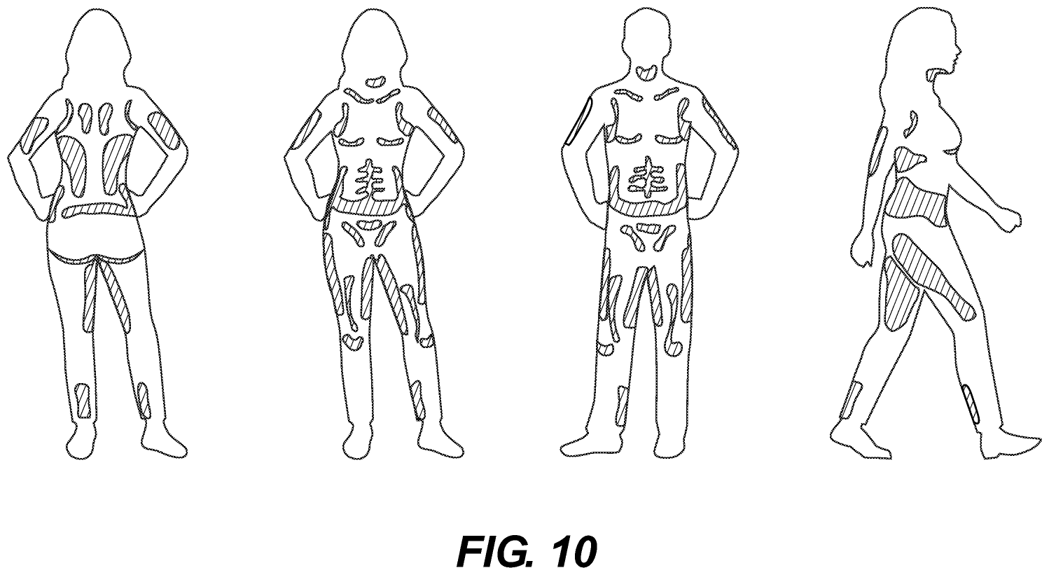

FIG. 9 shows an example point of delivery generation device having multiple cannulas or “working channels” to control the functions described above with reference to FIGS. 7A, 7B, and 8. The device includes an application cannula 705. The application cannula 705 houses a gas delivery cannula 710, a fluid delivery cannula 715, and a fluid return cannula 720, to continuously generate and replenish substantially solid solution, as described above with reference to FIGS. 7A and 8. The application cannula 705 can also include a retractable puncture needle 725 to rupture a balloon filled with substantially solid solution, as described above with reference to FIG. 7B. The application cannula 705 can further have a substantially solid solution temperature monitor 730 for measuring the temperature of the substantially solid solution.

An aspect of the invention includes a kit comprising one or more solutions comprising water and optionally one or more additives. When more than one solution is provided, the solutions may differ in their composition, including but not limited to the presence or absence of additives, the particular additives included and the amounts thereof, and the ice coefficient. The kit may also include one or more of a mold, a delivery device, a point-of-delivery generating device, a guide device, a balloon, a coil, a sterile bag (empty or pre-filled with a solution comprising water and optionally one or more additives), and a coating material.

The invention provides use of substantially solid solutions for a highly selective, precise, and targeted removal of fat in subcutaneous fat layers, resulting in superior results as compared to conventional methods. In some aspects, a slurry having an ice coefficient of approximately 2% to approximately 70% can be used alone or in combination with a substantially solid solution to treat adipose tissue such as slurry compositions described in International Application No. PCT/US19/54828 and methods and systems to generate a slurry described in International Application No. PCT/US19/55633, each incorporated herein by reference in their entirety. In this aspect, the slurry can comprise an ice coefficient of approximately 2-70%, for example, 20-50%, for example, 30-40%, for example, 31, 32, 33, 34, 35, 36, 37, 38, 39%.

The different properties present in superficial (sSAT) and deep (dSAT) layers of adipose tissue impact the activity of substantially solid solution compositions administered thereto. The sSAT and dSAT layers may be distinguished using standard ultrasound, as the fascial layer which separates the sSAT and dSAT layers is visible.

In an aspect of the invention, a determination is made regarding which layer of subcutaneous adipose tissue is to be treated, and the administration of the substantially solid solution injection to the desired layer is guided via ultrasound. Other imaging methods include, but are not limited to, Magnetic Resonance Imaging (MM), Computed Topography (CT), Positron Emission Tomography (PET), and combinations thereof.

In another aspect, a treatment specialist may rely upon variations in resistance when the delivery device, including but not limited to a syringe, a needle or a cannula, enters various layers of tissue, in order to determine proper placement within the appropriate layer. Less force may be necessary when administering the substantially solid solution into the sSAT layer as compared to the dSAT layer.

Aspects of the invention include administering a substantially solid solution into sSAT only, administering a substantially solid solution into dSAT only, administering a substantially solid solution into dSAT followed by administering into sSAT, administering a substantially solid solution into sSAT followed by administering into dSAT, and administering a substantially solid solution into dSAT and sSAT simultaneously. Therefore, aspects of the invention allow for selective targeting of sSAT and dSAT. In some aspects, administering a substantially solid solution into the dSAT followed by administering a substantially solid solution into the sSAT is utilized, in order to allow for visualization of each layer during the administration. In some aspects, multiple treatments may be performed, for example with a first session targeting the sSAT and a second session targeting the dSAT, or a first session targeting the dSAT and a second session targeting the sSAT. Any layer(s) can be treated in any order in any number of treatments.

To reduce pain associated with administration, methods of the invention may further comprise administering, topically and/or via injection, an anesthetic to an area for treatment of the subject prior to administration of the substantially solid solution. For example, the anesthetic may be a local anesthetic, including but not limited to lidocaine. In some aspects, the anesthetic may be administered to a subject a suitable amount of time in advance of the treatment in order to numb the administration area before treatment by the substantially solid solution.

The substantially solid solution is administered to a subject by any suitable method. In some aspects, the substantially solid solution is injected by any suitable means, such as injection by a cannula, a needle or a syringe and pump device. The needle may be any suitable type of surgical needle of any suitable size. In some aspects, the needle has a gauge size of about 8 G to about 25 G. In some aspects, the needle is a fenestrated needle.

In some aspects, the substantially solid solution is administered to a subject through an incision, wherein an incision is made at an appropriate location to allow for administration to the desired treatment site. One or more treatments may be administered through an incision, in successive administration, wherein each treatment may utilize a substantially solid solution with the same composition, or a different composition, depending upon a treatment plan created for the subject. The timing of the successive administrations may vary, depending upon the treatment plan.

In some aspects, the substantially solid solution is administered to a subject through a combination of injection and via an incision. Aspects of the invention include administering a substantially solid solution into sSAT via injection and incision, administering a substantially solid solution into dSAT via injection and incision, administering a substantially solid solution into dSAT via injection followed by administration into sSAT via incision, administering a substantially solid solution into dSAT via incision followed by administration into sSAT via injection, administering a substantially solid solution into sSAT via injection followed by administration into dSAT via incision, and administering a substantially solid solution into sSAT via incision followed by administration into dSAT via injection. In some aspects, multiple treatments may be performed, for example with a first session targeting the sSAT and a second session targeting the dSAT, or a first session targeting the dSAT and a second session targeting the sSAT. Any layer(s) can be treated in any order in any number of treatments.

Once the substantially solid solution is injected into a subject's body, the substantially solid solution causes cryolipolysis, or cell death by freezing of adipocytes. After cell death, the body naturally processes and eliminates the dead adipocytes.

Any suitable amount of substantially solid solution that is safe for administering to the subject may be administered. Different subjects have different amounts of subcutaneous fat. Therefore, some patients may require injection of greater amounts of substantially solid solution in order to produce visible effects of reduction and removal of subcutaneous fat. Other patients may require multiple treatments to produce the desired effects.

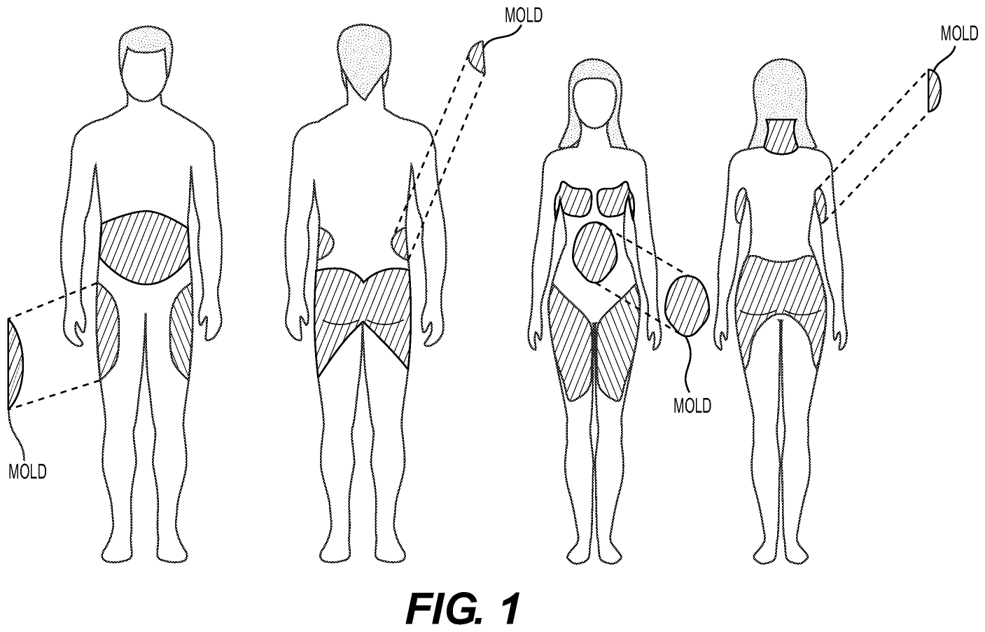

In some aspects, a treatment plan may include a combination of debulking, e.g., removal of large portions of adipocytes, and contouring, e.g., sculpting and/or shaping through discrete, targeted methods. For example, a subject seeking treatment of the abdominal area may benefit from a treatment plan which combines debulking and contouring. For the debulking, an incision may be made in the abdominal area, followed by administration of an ice composition formed in a shape appropriate for the treatment site. For example, see the mold shape provided in FIG. 1 for treatment of the abdominal area. Multiple administrations may be provided, until the desired results are achieved. Following the debulking, contouring may be performed by administering one or more ice needle compositions via injection or incision, in a pre-determined pattern, including but not limited to pre-determined intervals between muscle groups in order to remove discrete areas of adipocytes, thus forming the appearance of abdominal muscles, e.g., a “six-pack”. In some aspects, a slurry can be used in combination with a substantially solid solution, for example, the slurry can be used to debulk the abdomen, and the substantially solid solution can be used to contour the abdomen. In this aspect, the slurry can comprise an ice coefficient of approximately 2-70%, for example, 20-50%, for example, 30-40%, for example, 31, 32, 33, 34, 35, 36, 37, 38, 39%, and the substantially solid solution can comprise an ice needle composition. Conversely, a substantially solid solution can be used to debulk the abdomen, and a slurry can be used to contour the abdomen.

The substantially solid solution may also be administered with internal or external pressure on or near the treatment site to modify administration and/or effect of the substantially solid solution. For example, a balloon structure may be deployed at or near a point of delivery to act as an internal pressure device obstructing the flow of blood into a treatment area thus achieving extended cooling after administration. Approaches to delivery of a substantially solid solution utilizing balloon structures are disclosed, for example, in International Application Publication No. PCT/US2018/026273; U.S. Patent Application Publication No. 2018-0289538; and U.S. Provisional Application No. 62/482,008, which are incorporated herein by reference. In an embodiment, a vasoconstrictor is administered to the subject to reduce blood flow to achieve extended cooling. Pressure may also be applied externally using hand pressure and/or an applicator on the surface of the dermis. A negative pressure may also be applied for example, via a vacuum.

Any suitable amount of substantially solid solution that is safe for administering to the subject may be administered. For example, an amount of substantially solid solution to be administered by way of injection or via an incision may be selected based on subject characteristics, the treatment site and/or to the desired effects of treatment.

In aspects of the invention, an amount of substantially solid solution administered by injection may comprise about 2 L or less per injection site. In some aspects, an amount of substantially solid solution administered by injection may comprise about 1 mL to about 2 L per injection site, for example, about 2 mL, 3 mL, 4 mL, 5 mL, 10 mL, 50 mL, 1 L, or about 1.5 L. Different subjects may have different amounts of subcutaneous fat, and accordingly, some subjects may require administration of larger amounts of substantially solid solution in order to produce visible effects of reduction and removal of subcutaneous fat, and/or tightening of the skin as a result of a collagen response. Some subjects may require multiple treatments to produce effects of removal or reduction of subcutaneous fat and/or tightening of the skin as a result of a collagen response.

The amount of water in a solid state in the substantially solid solution to be administered may vary, based upon the treatment plan. In one aspect, the substantially solid solution may be administered to target a large area, such as the abdomen, utilizing a large volume of substantially solid solution and/or an ice composition having an ice coefficient of about 95 to about 100%. In another aspect, the substantially solid solution may be administered to target a small area, such as the chin, utilizing a smaller volume of substantially solid solution and/or utilizing a substantially solid solution with a lower ice content, such as 71%.

Treatment may also comprise tightening of skin of the subject. The tightening of the skin results from a collagen response upon removal and reduction of the fat cells in the subcutaneous fat layer. Reduction of subcutaneous fat may also reduce adipose tissue hypoxia or inflammatory signaling in overweight and obese subjects. Additionally, the substantially solid solution may also be utilized to mechanically disrupt fibrous tissue to break up compartments found within the subcutaneous fat, allowing the subcutaneous fat to spread and create a visually smoother appearance, for example in the treatment of cellulite.

In another aspect of the invention, a device is generated by placing one or more resorbable sutures in the selected mold, for example, a cannula, and placing the solution comprising water and optionally one or more additives in the mold, prior to cooling. After achieving the desired ice coefficient through cooling, the substantially solid solution further comprising one or more resorbable sutures is removed from the mold in a manner described previously herein, and administered to the desired treatment site through an appropriate method, for example, via injection. After administration, the substantially solid solution melts, through any suitable active or passive measure. The one or more resorbable sutures remain at or near the treatment site until resorption acting as an irritant to the tissue surrounding the treatment site. A cosmetic benefit occurs due to the increased collagen produced around the treatment site due to the presence of an unknown irritant, specifically the one or more resorbable sutures. The increased collagen promotes cell renewal, which provides cosmetically appealing results. In some aspects, the sutures may be provided in a pattern suitable to the treatment site. For example, when treating the chin and neck area, the sutures may be administered in an arc pattern just below the jawline, thus providing minimally visible suture sites.

Treatment with the substantially solid solution may be optimized for cosmetic or aesthetic results, for example to achieve smoothing and to avoid the appearance of sharp edges in the subcutaneous layer or layers. In some aspects, a profile can be created that correlates to the ice coefficient of the substantially solid solution. For example, a substantially solid solution with a higher ice coefficient may be used to treat the center of a treatment site, while a substantially solid solution with a lower ice coefficient may be used to treat the outer perimeter of the treatment site. Any of the substantially solid solution properties such as ice coefficient, size and shape, may be varied to achieve a desired result.

In an aspect of the invention, a treatment plan may be created for a subject, for example to determine the substantially solid solution properties, amount of substantially solid solution to be delivered, treatment sites such as superficial and/or deep layers, and the most suitable device and method for administration of the substantially solid solution. Factors considered in creating a treatment plan for a subject may comprise one or more of gender, height, body weight, body fat percentage, anatomy such as septae rigidity, lifestyle, vitals, medical history, lipid profiles, skin elasticity, medication, nutrition, supplements, demographic, vascularity of fat tissue, fat saturation, and the like. Fat saturation may be characterized by one or more of imaging, biopsy, and impedance measurement. In some aspects, after a plan is created for the subject, the amount of substantially solid solution to be administered can be adjusted based on one or more of the area or areas to be treated, the subcutaneous fat layers to be treated, the depth of injection, and the injection pattern to be used.

A computer or artificial intelligence system may be utilized to create a treatment plan for a subject by collecting pre-, peri-, and/or post-injection data from multiple subjects. It is appreciated that the more data points, the more effective the artificial intelligence system will be in creating a treatment plan for a subject. For example, pre-, peri-, and/or post-injection data may be collected for each subject comprising one or more of gender, height, body weight, body fat percentage, the subject's anatomy such as septae rigidity, lifestyle, the subject's vitals, medical history, lipid profiles, skin elasticity, medication, nutrition, supplements, demographic, fat saturation, imaging data, treatment data and fat loss data. Data may be measured by any suitable means. For example, fat loss data may be measured by calipers or any imaging methods, including but not limited to ultrasound and MRI.

Imaging may also be utilized during creation of a treatment plan for a subject by collecting pre-, peri-, and/or post-injection data from one or more subjects. Information may be obtained through any suitable procedure, including but not limited to Magnetic Resonance Imaging (MRI), Computed Topography (CT), ultrasound, Positron Emission Tomography (PET), and combinations thereof. Utilizing images of the treatment site, surrounding areas, and/or other areas of interest, may provide detailed information regarding the most suitable treatment plan.

In an aspect of the invention, an ice composition may be generated with a hollow core, for example, a hollow cylindrical ice composition. In another aspect, the ice composition with a hollow core may include one or more mechanical latches, wherein the ice composition may be injected into the subject in an area where diagnostic analysis of tissue is needed. The injection of the ice composition with a hollow core allows collection of a tissue sample in the hollow core, wherein the one or more mechanical latches opens upon injection, and closes upon collection of the tissue sample. The tissue sample is then extracted from the subject upon removal of ice composition. The cold temperature of the hollow cylindrical ice composition maintains the integrity of the tissue sample during extraction. Additionally, the cold temperature of the hollow cylindrical ice composition may be actively or passively melted after extraction and removal of the tissue sample, thus allowing ease of diagnostic analysis of the obtained tissue sample.

In another aspect, an ice composition with a hollow core may be administered to the subject, followed by administration of a substantially solid solution through the hollow core, wherein the substantially solid solution has a lower ice coefficient than the ice composition with a hollow core. In another aspect, an ice composition with a hollow core may be administered to the subject, followed by administration of a slurry through the hollow core, wherein the slurry has a lower ice coefficient than the ice composition with a hollow core. In some aspects, where a treatment plan requires simultaneous administration of two substantially solid solutions having different ice coefficients, after administration of the substantially solid solution through the hollow core of the ice composition, the ice composition may be broken in place through any suitable method, including but not limited to mechanical vibration, manual pressure to the treatment site, or use of a guide device.

A substantially solid solution may be injected at multiple treatment sites, wherein the selected treatment sites may be the superficial layer (sSAT), the deep layer (dSAT), or both. The substantially solid solution may be injected into sSAT and/or dSAT at a plurality of injection sites. The injection sites may form a pattern, such as a plow, fan, or grid-like pattern, or may be a single bolus or multiple bolus injections. A pre-fabricated needle array may be utilized to simultaneously administer the substantially solid solution to multiple injection sites. The pre-fabricated array may be formed based upon the specific administration site, taking into consideration the size and shape of the administration site, as well as the desired number of administrations. One injection site may be used repeatedly, thereby reducing the number of injection sites and associated potential for scarring. In a plow injection pattern, a single initial target injection site may be used, followed by a moving needle for additional deposition sites, for example in a linear pattern. In a fan injection pattern, deposition sites may form an arc from 1 to 360 degrees. In a bolus injection, the substantially solid solution is deposited in a single injection site. The deposition site is where the substantially solid solution is deposited, regardless of the injection site, and may be a different site than the injection site or the same site.

The injection pattern can be determined based on the subject's profile, treatment plan, or based on the target site to be treated. An injection pattern and/or volume may be selected to optimize consistency of temperature at the target site. The injection pattern and/or volume may be selected in order to achieve gradient cooling of fat proximate to a target site or injection site. Injection techniques, including the patterns described herein, are known to those of skill in the art.

In some aspects, the superficial layer (sSAT) is treated at the same time as the deep layer (dSAT). For example, the substantially solid solution may be injected into superficial subcutaneous fat, and then the delivery device, e.g., needle, is moved deeper in the deep subcutaneous fat regions. In an aspect of the invention, a fenestrated needle having a suitable length with fenestrations in both the first subcutaneous fat layer and the second subcutaneous fat layer is used to treat the first and second subcutaneous fat layers at the same time. In another aspect, the superficial subcutaneous fat layer and the deep subcutaneous fat layer are treated at the same time by injecting a needle and slowly withdrawing the needle, releasing substantially solid solution in both subcutaneous fat layers.

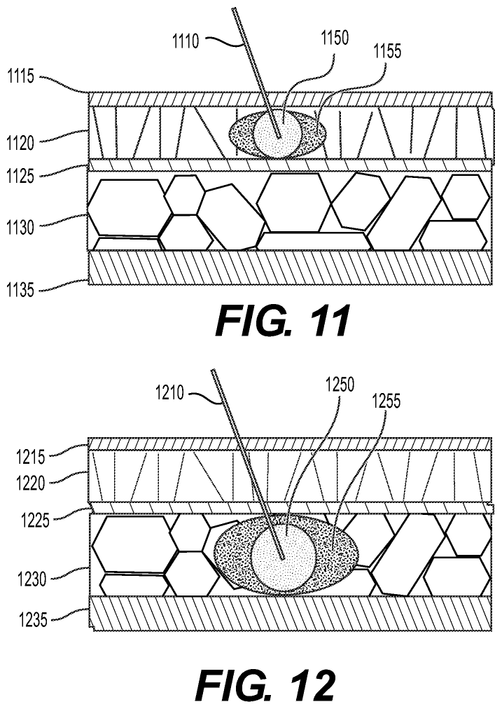

FIG. 11 shows injection of a substantially solid solution into superficial fat (sSAT) 1120. The substantially solid solution 1150 is at the injection site, and as the substantially solid solution melts, a liquid component 1155 of the substantially solid solution expands from the injection needle 1110. The sSAT 1120 is bounded superiorly by the skin 1115 and inferiorly by the fascia 1125. The deep fat layer (dSAT) 1130 is bounded superiorly by the fascia 1125 and inferiorly by muscle 1135. Similarly, FIG. 12 shows injection of a substantially solid solution into dSAT 1230. The substantially solid solution 1250 is at the injection site, and as the substantially solid solution melts, a liquid component 1255 of the substantially solid solution expands from the injection needle 1210. The sSAT 1220 is bounded superiorly by the skin 1215 and inferiorly by the fascia 1225. The dSAT 1230 is bounded superiorly by the fascia 1225 and inferiorly by muscle 1235.

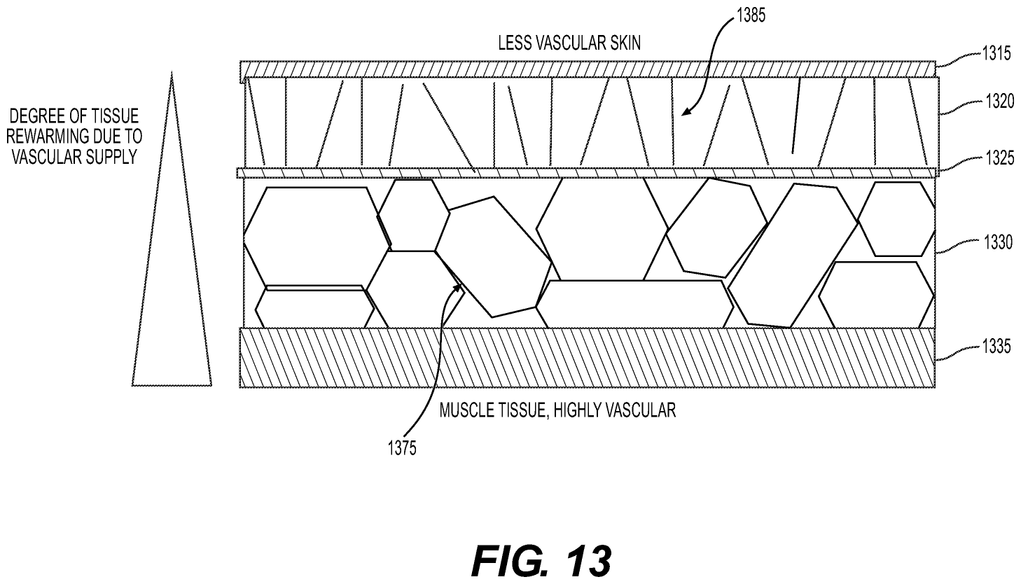

In another aspect, in order to obtain equivalent cooling durations, it may be necessary to inject a greater volume into the dSAT compared to the sSAT. Given the increased radial spread of a fixed volume in the dSAT, the concentration of solid water may be less dense and melt more quickly than the more densely packed solid water in the sSAT. Additionally, dSAT is closer to highly vascular underlying muscle. The proximity to underlying muscle may cause the area to rewarm more quickly than the less vascular sSAT that is further from muscle. FIG. 13 shows the degree of tissue rewarming due to vascular supply which decreases when traveling upwards from the highly vascular muscle tissue towards the less vascular skin. In relation, dSAT has a greater relative vascularity than sSAT.

The lamellar pattern of the sSAT is shown in FIG. 13, as well as the loose areolar pattern of dSAT. The sSAT 1320 consists of regular, defined cuboid fat lobules tightly organized within vertically oriented fibrous septae 1385. The dSAT 1330 has a loose areolar pattern with fat lobules 1375 that are flat shaped, irregular in size, and are surrounded by high amounts of loose connective tissue. The skin 1315, fascia 1325, and muscle 1335 are also designated.

In another aspect, shown in FIG. 14, sSAT can be selectively targeted with a substantially solid solution 1460 to target and remove sSAT. The substantially solid solution 1460 is injected in the sSAT 1420 located below the skin 1415 and above the fascia 1425. The dSAT 1430 is shown between the fascia 1425 and the muscle 1435.

In another aspect, shown in FIG. 15, dSAT can be selectively targeted with a substantially solid solution 1560 to target and remove dSAT. The substantially solid solution 1560 is injected in the dSAT 1530 located between the muscle 1535 and the fascia 1525. The dSAT 1530 sits below the sSAT 1520, which is located between the fascia 1525 and the skin 1515.

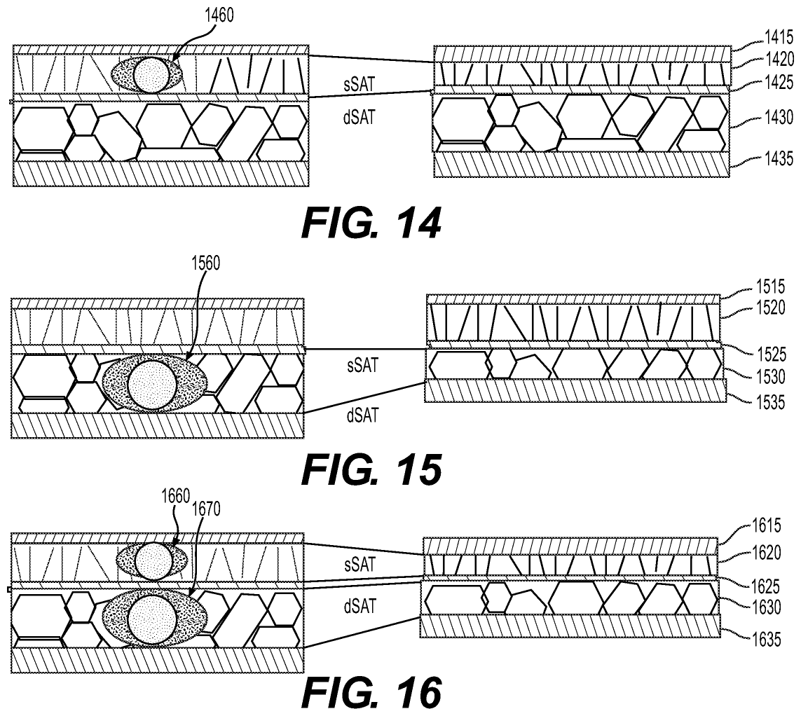

In another aspect, shown in FIG. 16, both sSAT and dSAT can be selectively targeted with substantially solid solutions 1660 and 1670 to target and remove both dSAT and sSAT. The substantially solid solution 1660 is injected in the sSAT 1620 located below the skin 1615 and above the fascia 1625. The substantially solid solution 1670 is injected in the dSAT 1630 located between the muscle 1635 and the fascia 1625. Substantially solid solution 1660 and substantially solid solution 1670 may be the same substantially solid solution or may be different substantially solution solutions.

Targeting of the respective subcutaneous fat layer may occur by localizing substantially solid solution injections to that layer (or sublayer or compartments within a layer). Further, reducing or removing the fat cells may result in a collagen response, such as shown by thickening of the skin in FIG. 14 and FIG. 16. The collagen response works to tighten the skin in the treated area.

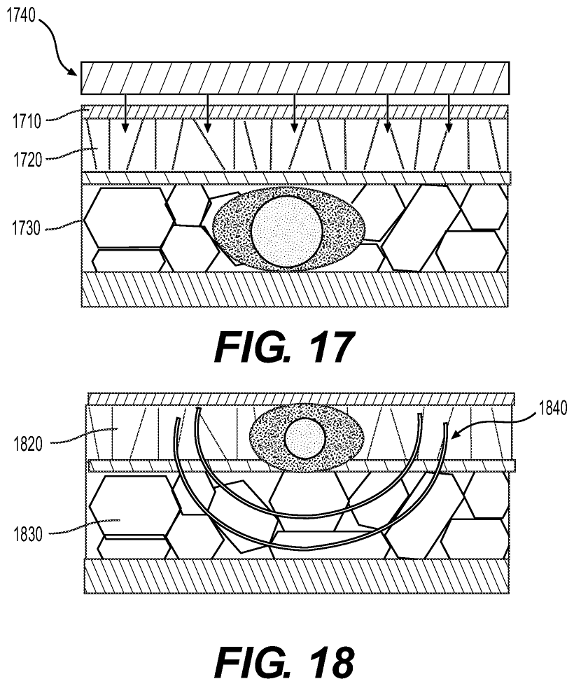

In addition to achieving selectivity by localizing substantially solid solution injection, selectivity can be achieved or augmented by combining an injection with active warming of the layers that are not targeted. For example, as shown in FIG. 17, to limit substantially solid solution cooling to only dSAT 1730, sSAT 1720 could be actively warmed through application of a heating source 1740 to the skin 1710, or by use of infrared radiation. Conversely, as shown in FIG. 18, to selectively target sSAT 1820, the dSAT 1830 could be actively warmed using a modality such as radiofrequency 1840.

FIGS. 11-18 illustrate administration of the substantially solid solution via injection, however, the substantially solid solution can be administered via incision in either or both of the sSAT and/or dSAT.

Since the anatomy of fat layers differ, not all body areas containing excess unwanted subcutaneous fat are suitable for treatment of sSAT and dSAT. The body areas that contain dSAT also contain a sSAT layer, making these areas suitable for both depths of treatment, whereas many body areas only have a sSAT layer. For example, submental, upper arm, lower thigh medially or laterally, supragenicular, ankle, or facial fat only contain sSAT. Other areas, such as abdomen, flank, lumbar or buttock fat are suitable to both dSAT and sSAT targeting.

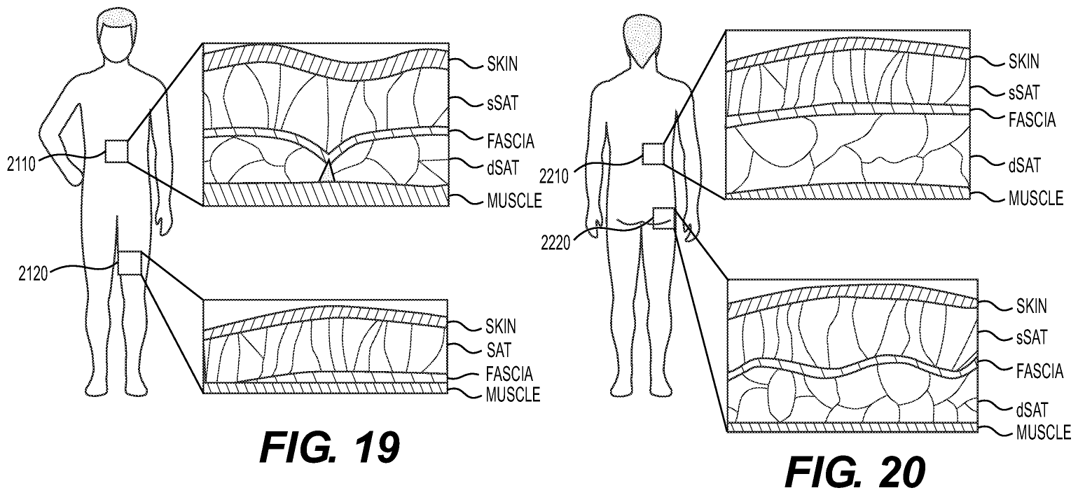

FIG. 19 shows variations in fat by anatomic area, particularly the abdomen 2110 and lower thigh 2120 areas. FIG. 20 shows variations in fat by anatomic area, particularly the back 2210 and buttocks 2220.

Pre- or post-administration steps may also be utilized to optimize substantially solid solution treatment results. In one aspect, a massaging step may be utilized to increase fat cell damage and/or the mechanical force of the water in solid state in the substantially solid solution. The massaging may be performed to puncture one or more cell membranes. The massaging step may be used to position or shape the substantially solid solution post administration. Massaging can be performed by any mechanical means, for example by hand, vibration, an applicator, or by acoustic means. Additionally or alternatively, the substantially solid solution may be broken in place through any suitable method, including but not limited to mechanical vibration, manual pressure to the treatment site, or use of a guide device.

The substantially solid solutions and methods described above can be provided to a tissue within the body of a patient, for example, for the treatment of a patient. The tissue to which the substantially solid solution can be administered includes one or more of connective, epithelial, neural, joint, cardiac, hepatic, renal, vascular, cutaneous, and muscle tissue. Additionally, methods include delivery of one or more substantially solid solutions to any one or more of the following locations: proximate to a nerve, proximate to subcutaneous adipose tissue, proximate to breast tissue, proximate to visceral fat, fatty tissue proximate to the pharynx, fatty tissue proximate to the palate, fatty tissue proximate to the tongue, proximate to a spinal cord lipoma, proximate to visceral fat, proximate to lipomastia, proximate to a tumor, proximate to cardiac tissue, proximate to pericardial fat, proximate to epicardial fat, proximate to a lipid-rich plaque in the vasculature, and proximate to areas of steatosis or ectopic fat in muscle.

Various conditions, disorders, or diseases which can be treated through delivery of a therapeutically effective amount of one or more substantially solid solutions to a subject include excess fat, obesity, loose skin, metabolic dysfunction, insulin resistance, type II diabetes, systemic inflammation, inflammatory disorders, hypertension, hyperlipidemia, elevated triglycerides, dyslipidemia, fatty liver disease, obstructive sleep apnea, lipedema, lymphedema, non-alcoholic steatohepatitis, atrial fibrillation, atherosclerosis, systemic inflammation, inflammatory disorders, nerve pain, lipodystrophy, decrum's disease, lipomatosis, familial multiple lipomatosis, aberrant fat tissue proliferation, Proteus syndrome, Cowden Syndrome, Modeling disease (benign symmetric lipomatosis), familial angiolipomatosis, lymphatic leakage, de novo adipogenesis, increase in adipocyte cell size, and adipocyte proliferation due to excessive leakage of lymphatic including leakage of free fatty acids containing lymph.

After completion of treatment, the subject may be monitored. Additionally, data may be obtained from the subject for post-treatment analysis, which may include utilization of a computer or artificial intelligence system. The data may include gender, height, body weight, body fat percentage, the subject's anatomy such as septae rigidity, lifestyle, the subject's vitals, medical history, lipid profiles, skin elasticity, medication, nutrition, supplements, demographic, fat saturation, imaging data, treatment data and fat loss data. Data may be measured by any suitable means, including but not limited to inquiries, measurements, imaging and visual analysis. The obtained data may be compared to data obtained prior to, and during, the treatment plan, and may be used to determine the effectiveness of the treatment, and/or whether additional treatments are desired.

In some aspects, the substantially solid solution, for example an ice needle composition, can be coated with an agent such as a biocompatible and biodegradable material such as a naturally occurring polymer. In some aspects, the agent can be inert to improve mechanical properties such as ease of injection or rate of thermal transfer as described above. In some aspects, the agent can comprise a pharmacologic agent that acts locally as it releases over time. Examples include an anesthetic/analgesic to address pain, an anti-inflammatory agent to decrease post procedure swelling, a vasoconstrictor to prolong cooling or augment fat removal via ischemia and/or decrease risk of hematoma/bruising, a lipolytic agent to augment fat removal, an agent known to increase collagen production or components of the extracellular matrix to enhance tightening effects, or an agent known to activate brown or beige fat such as β3-adrenergic receptor agonist, PPAR-γ activators, PGC-1α stabilizers, PPAR-α agonist, AMPK activators and PDE5 inhibitors such as Sildenafil and Tadalafil. In some aspects, an ice needle composition can be configured as a drug delivery device. In these aspects, an agent can be encapsulated, for example in nanoparticles or microparticles, that are frozen in the ice needle composition, for example in the mold, and are released into tissue as the ice needle composition melts. Similarly, an ice needle composition can be used to deliver larger molecules such as proteins and large peptides that have functions similar to the above.

All documents, books, manuals, patents, published patent applications, and other reference materials cited herein are incorporated by reference in their entirety.

While the invention has been described with reference to certain particular aspects thereof, those skilled in the art will appreciate that various modifications may be made without departing from the spirit and scope of the invention. The scope of the appended claims is not to be limited to the specific embodiments described.

What is claimed is:

1. A composition comprising liquid water, about 71% to about 100% by volume of water in a solid state, and optionally one or more additives.

2. The composition of claim 1, comprising about 95% to about 100% by volume of water in a solid state.

3. The composition of claim 2, comprising about 99% to about 100% by volume of water in a solid state.

4. The composition of claim 1, comprising at least one additive selected from the group consisting of a salt, a sugar and a thickener.

5. A method for treatment of subcutaneous adipose tissue, said method comprising:
administering the composition of claim 1 to a treatment site of a subject, wherein the treatment site is selected from the group consisting of (i) superficial subcutaneous adipose tissue, (ii) deep subcutaneous adipose tissue, and (iii) superficial subcutaneous adipose tissue and deep subcutaneous adipose tissue.

6. The method of claim 5, wherein the composition is administered to superficial subcutaneous adipose tissue, followed by administration to deep subcutaneous adipose tissue.

7. The method of claim 5, wherein the composition is administered to deep subcutaneous adipose tissue, followed by administration to superficial subcutaneous adipose tissue.

8. The method of claim 5, wherein the composition is administered to deep subcutaneous adipose tissue and superficial subcutaneous adipose tissue simultaneously.

9. The method of claim 5, wherein the composition is administered to a plurality of treatment sites.

10. The method of claim 5, wherein the composition is injected into the treatment site of the subject.

11. The method of claim 8, wherein the composition is injected into the plurality of treatment sites, through a plurality of injection sites.

12. The method of claim 11, wherein the plurality of injection sites forms a pattern.

13. The method of claim 12, wherein the pattern is a plow pattern, a grid pattern, or a fan pattern.

14. The method of claim 5, wherein an incision is made to allow access to the treatment site, and the composition is administered by insertion at the treatment site of the subject through the incision.

15. The method of claim 10, wherein the composition is injected into the treatment site using a needle.

16. The method of claim 11, wherein the composition is injected into the plurality of treatment sites using one or more needles.

17. The method of claim 15, wherein the needle comprises a gauge size of about 8 G to about 25 G.

18. The method of claim 16, wherein the one or more needles comprise a gauge size of about 8 G to about 25 G, and the gauge size of each needle may be the same or different.

19. The method of claim 10, wherein the composition is injected into the treatment site using a cannula.

20. The method of claim 11, wherein the composition is injected into the plurality of treatment sites using one or more cannulas.

21. The method of claim 5, wherein the treatment of subcutaneous adipose tissue comprises initiating cryolipolysis.

22. The method of claim 21, wherein the treatment of subcutaneous adipose tissue further comprises tightening of the subcutaneous adipose tissue.

23. The method of claim 5, further comprising administering an anesthetic to the subject prior to and/or during the administration of the composition.

24. A method of creating a treatment plan for a subject, said method comprising obtaining information from the subject prior to, during, and/or after administration of the composition of claim 1, providing the information to a computer or artificial intelligence system, and utilizing the computer or artificial intelligence system to create the treatment plan for the subject.

25. The method of claim 24, wherein the information obtained from the subject comprises one or more of gender, height, body weight, body fat percentage, septae rigidity, lifestyle, vitals, medical history, lipid profiles, skin elasticity, medication, nutrition, supplements, demographic, vascularity of fat tissue, and fat saturation.

26. The method of claim 24, wherein the information is obtained from the subject using imaging.

27. The method of claim 5, wherein the method further comprises treatment of a condition, disorder or disease by administration of a therapeutically effective amount of the composition to the subject, wherein the condition, disorder or disease is one or more of excess fat, obesity, loose skin, metabolic dysfunction, insulin resistance, type II diabetes, systemic inflammation, inflammatory disorders, hypertension, hyperlipidemia, elevated triglycerides, dyslipidemia, fatty liver disease, obstructive sleep apnea, lipedema, lymphedema, non-alcoholic steatohepatitis, atrial fibrillation, atherosclerosis, systemic inflammation, inflammatory disorders, nerve pain, lipodystrophy, decrum's disease, lipomatosis, familial multiple lipomatosis, aberrant fat tissue proliferation, Proteus syndrome, Cowden Syndrome, Modeling disease (benign symmetric lipomatosis), familial angiolipomatosis, lymphatic leakage, de novo adipogenesis, increase in adipocyte cell size, and adipocyte proliferation due to excessive leakage of lymphatic including leakage of free fatty acids containing lymph, and wherein the subject is in need of treatment of the condition.

28. A method for generating a composition comprising liquid water, about 71% to about 100% by volume of water in a solid state, and optionally one or more additives, said method comprising cooling a composition comprising liquid water and the optionally one or more additives until about 71% to about 100% by volume of water in a solid state is obtained.

29. The method of claim 28, wherein the cooling is achieved by freezing or supercooling the composition comprising liquid water and optionally one or more additives.

30. A method for generating a composition comprising liquid water, about 95% to about 100% by volume of water in a solid state, and optionally one or more additives, said method comprising cooling a composition comprising liquid water and the optionally one or more additives until about 95% to about 100% by volume of water in a solid state is obtained.

31. The method of claim 29, further comprising placing the composition comprising liquid water and the optionally one or more additives in a mold prior to freezing or supercooling.

32. The method of claim 30, wherein the mold is in a shape suitable for a desired treatment site.

33. The method of claim 32, wherein the mold shape is a cylinder, a cube, a cuboid, a sphere, a rounded mold, a triangular prims, a cone, or a combination thereof, wherein the shape may include variance.

34. The method of claim 32, wherein the mold shape is a unique shape.

35. The method of claim 32, wherein the mold comprises an open structure.

36. The method of claim 32, wherein the mold comprises a closed structure.

37. The method of claim 31, wherein the mold is a cannula or a needle.

38. The method of claim 31, wherein the mold has a selected size and a selected shape, wherein the selected size and the selected shape are determined through analysis of the treatment site.

39. The method of claim 38, wherein the analysis of the treatment site is conducted by measuring the treatment site, an area surrounding the treatment site, or a combination thereof.

40. The method of claim 39, wherein the measuring is conducted through visual measuring, measuring using imaging, computer assisted measuring, artificial intelligence assisted measuring, or a combination thereof.

41. The method of claim 31, wherein the mold is made of any suitable material.

42. The method of claim 5, further comprising administering a slurry to a treatment site of a subject.
